(19) United States
(12) Patent Application Publication
(10) Pub. No.: US 2021/0381067 A1
(43) Pub. Date: Dec. 9, 2021

(54) NUCLEIC ACID AMPLIFICATION AND DETECTION WITH ATTENUAITING PROBE

(71) Applicant: Ampliwise Inc., Santa Clara, CA (US)

(72) Inventors: Xing SU, Cupertino, CA (US); Kai WU, Mountain View, CA (US)

(21) Appl. No.: 16/930,426

(22) Filed: Jul. 16, 2020

Related U.S. Application Data
(60) Provisional application No. 63/036,076, filed on Jun. 8, 2020.

(51) Int. Cl. C12Q 1/70

(52) U.S. Cl. CPC C12Q 1/701

(57) ABSTRACT

An embodiment relates to a method comprising assembling a reaction mixture comprising: a target molecule comprising a nucleic acid sequence of interest; a set of oligonucleotides comprising: a 1^{st} SW (selective wobble) primer comprising a 1^{st} SW site; a 2^{nd} SW primer comprising a 2^{nd} SW site; at least a third primer; a probe comprising: (i) an attenuating site, (ii) a first label in a non 3′ site and (iii) a second label at the 3′ end; a polymerase with 3′-5′ exonuclease activity; conducting an amplification reaction of the target molecule comprising the nucleic acid sequence of interest using the reaction mixture; detecting or amplifying the target molecule comprising the nucleic acid sequence of interest or variants thereof present in the target molecule, wherein the SW sites are configured to enable non-disrupted nested amplification and quantification of the target molecule comprising the nucleic acid sequence of interest. The nucleic acid sequence of interest comprises a SARS-CoV-2 sequence.

### RELATED APPLICATIONS

This application claims the benefit of priority under 35 U.S.C. § 119(e) from U.S. Provisional Application No. 63/036,076, filed on Jun. 8, 2020, which is related to U.S. patent application Ser. No. 15/597,310, filed on May 17, 2017, entitled “COMPOSITIONS AND METHODS FOR NUCLEIC ACID AMPLIFICATION” which is incorporated herein in its entirety by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: AMPL-005-01US_ST25, date recorded: Oct. 28, 2020, file size 3 kilobytes).

### FIELD OF THE INVENTION

The invention relates to methods for amplifying and/or quantifying target nucleic acid molecules of interest, more particularly, to a method of amplifying and detecting a target nucleic acid of interest via a novel PCR probe containing an attenuating site and a single tube nested polymerase chain reaction (PCR). The embodiments of present disclosure further relate to compositions, kits, probe and primer set for detecting and/or quantifying target nucleic acid molecules of interest.

### BACKGROUND OF THE INVENTION

The polymerase chain reaction (PCR) is a revolutionary method developed by Kary Mullis in the 1980s (Mullis K et al., 1986) and is one of the most powerful technologies in molecular biology. Using PCR, a specific sequence within a DNA or cDNA molecule or a nucleic acid template can be amplified from small amounts to many thousand- to a million-fold using sequence-specific primers, heat-stable DNA polymerase, and thermal cycling. The advantage of PCR is that it amplifies small amounts of nucleic acids by million- to billion-fold (Glennon M. and Cormican M., 2001). One of the major applications of PCR is as a research or diagnostic tool, for example, in detecting the presence of pathogenic virus or bacteria (Yamamoto Y. 2002).

Although PCR is time-saving and a relatively sensitive diagnostic tool, however, false negative signals often arise in samples with a very low copy number of a nucleic acid sequence (Bonne et al., 2008). This becomes problematic when the pathogen for an outbreak of a disease, such as COVID-19, or infection needs to be detected at the very early stages of infection, in order to prevent and contain the spread of the infection or disease. Another problem with conventional PCR technique is primers binding to incorrect regions of the DNA, giving false positive products. This problem becomes more likely with an increased number of cycles of PCR.

Real-time reverse transcription (RT)-PCR detection has been used for the detection of pathogens (Drosten C S et al., 2003) and recently for the detection of SARS-CoV-2 that is the causative agent for COVID-19. “Real-time” detection allows one to measure the accumulation of PCR product during the course of the reaction, rather than simply analyzing the final product amount following the course of sequential cycles of amplification (Poon L L et al., 2003).

Presently, qPCR assay is based on the hybridisation of a dual-labeled probe to the PCR product, for example the so-called “TaqMan probes”, in which the development of a signal results from the loss of fluorescence quenching in the probe (Ponchel F et al., 2003). The TaqMan probe principle relies on the 5′-3′ exonuclease activity of the Taq DNA polymerase to cleave a dual-labeled probe during hybridization to the complementary target sequence and thus release the fluorophore from the probe for fluorescence detection. However, the 5′-3′ exonuclease activity of the Taq DNA polymerase is very weak and requires strong binding of the TaqMan probe to the nucleic acid sequence for efficient signal generation (Tajadini M et al., 2014). The strong binding can slower down primer extension and thus reduce the rate and yield of the PCR reaction. This qPCR assay is generally very inconsistent in the clinical diagnostic setting. False-negatives due to lack of sensitivity of assays may mislead clinicians to discharge early infected individuals from hospitals.

Toward the goal of reducing diagnostic false positives or false negatives while retaining high sensitivity and specificity, a new PCR probe has been developed that takes advantage of the 3′-5′ exonuclease activity of many DNA polymerases, instead of the 5′-3′ exonuclease activity in Taq DNA polymerase. DNA polymerases with 3′-5′ exonuclease activities are superior to Taq DNA polymerase because they are generally more thermal stable and have much high fidelities. However, these enzymes cannot be used for reliable quantitative PCR using the same probe design as the TaqMan probe. The reason is that a probe (a labeled oligonucleotide) can be extended if a terminal nucleotide has a free hydroxyl group at its 3′ end after exonuclease reaction, resulting in non-specific signals if the extension happens to non-target template molecules. This problem can even be more pronounced if the nonspecific extension leads to a subsequent non-specific amplification. We have also developed a new nested PCR procedure for detecting low-copy-number of nucleic acids molecules. Nested PCR is a modification of PCR that is designed to improve sensitivity and specificity. A typical nested PCR involves the use of two primer sets and two successive PCR reactions (Grünebach F et. al., 1994). The first set of primers are designed to anneal to sequences outside (outer) from the second set of primers (inner) and are used in an initial PCR reaction. Amplicon products resulting from the first round of PCR reaction are used as templates for a second set of primers and a second round of amplification (Zeaiter Z et al. 2003). Sensitivity and specificity of DNA amplification is significantly enhanced with this technique (Kim D M et al., 2011).

A nested PCR is conventionally performed by carrying out an initial PCR in one reaction tube, transferring an aliquot of the amplified products into a second reaction tube, and then carrying out a second PCR. This procedure has two disadvantages. It is more complex than a single PCR and, more importantly, it carries the risk of contaminating the environment with the amplified products of the first PCR, which may lead to contamination of subsequent experimental procedures. Single tube nested PCR, unlike the two-steps nested PCR, is less complex to operate, and eliminates chance to create amplicon contamination.

Therefore, there is a need to develop a new PCR probe method for specific and quantitative nucleic acid detection and a single-tube nested qPCR which is convenient to use, less chance to create amplicon contamination, shorter time to complete and cost-effective. This novel technique combines the high sensitivity of nested PCR with the specificity and/or quantification of novel probe will improve detection of nucleic acid target of interest, such SARA-CoV-2 the causative agent of COVID-19.

### SUMMARY OF THE INVENTION

Embodiments relate to a method for amplification and detection of a target nucleic acid sequence by the single-tube nested PCR with an increase in sensitivity, target specificity, and a decrease in cross-sample contamination. Further, an embodiment relates to a method for quantifying a target nucleic acid sequence. The method comprises subjecting target nucleic acid sequences to single-tube nested qPCR.

An embodiment relates to a specific and/or quantitative detection of the nucleic acid sequence of interest. A method comprising: (a) assembling a reaction mixture comprising: (i) a target molecule comprising a nucleic acid sequence of interest; (ii) a primer set comprises a pair of amplification primers; (iii) a probe comprising: (1) an attenuating site, (2) a first label in a non 3′ site and (3) a second label at the 3′ end; (iv) a polymerase with a 3′-5′ exonuclease activity; (b) conducting an amplification reaction of the target molecule comprising the nucleic acid sequence of interest using the reaction mixture. The probe and the polymerase with 3′-5′ exonuclease activity is configured to enable the specific and/or quantitative detection of the nucleic acid sequence of interest. In one embodiment, the nucleic acid sequence of interest comprises a SARS-CoV-2 sequence or its derivative sequences.

In an embodiment, the second label at the 3′ end of the probe is cleavable effectively using the 3′-5′ exonuclease activity of the polymerase.

In an embodiment, the first label and the second label comprise a fluorescent dye-quencher pair or similar thereof.

In an embodiment, the primer set comprises a first primer and a second primer, wherein the first primer and the second primer are complementary to the target molecule comprising the nucleic acid sequence of interest.

In an embodiment, the primer set comprises a first primer (forward) and a second primer (reverse), wherein the first primer and the second primer are complementary to the target molecules comprising the nucleic acid sequences of interest. The target molecules could be single-stranded or double-stranded nucleic acid molecules. The target molecules could be RNA or DNA. In an embodiment, the nucleic acid sequence of interest is a sequence derived from SARS-CoV-2 genome RNA sequence. The derived sequence can be a fragment, a region of the genomic RNA sequence. The derived sequence can also be a cDNA sequence that is reverse transcribed from the genomic RNA sequence RNA. The reverse transcription (RT) can take place in the same tube prior to the PCR reaction (e.g., including nested PCR, probed-PCR, quantitative PCR, or combination thereof); thus a RT-PCR can be performed in the same tube, using the same reaction mixture for the detection of a pathogen, such as SARS-CoV-2 virus that is the causative agent for COVID-19.

In an embodiment, the attenuating site is located between the center of the probe and the second label and comprises at least 1 to 10 units, comprising a natural nucleotide, a non-natural nucleotide, an abasic site, a spacer, a fluorescent label-modified nucleotide, an atypical nucleotide comprised of deoxyuridine, a chemically synthesized nucleotide or combination thereof.

In an embodiment, the attenuating site comprises at least 1 to 10 units. A unit in the attenuating site is a nucleotides or nucleotide-equivalent in a polynucleotide molecule. The attenuating site comprises a structure comprising a natural nucleotide, a non-natural nucleotide, an abasic site, a spacer, a fluorescent label-modified nucleotide, an atypical nucleotide comprised of deoxyuridine, a chemically synthesized nucleotide or combination thereof. The attenuating site is located between the center of the probe and the second label. It is configured to prevent the probe molecules from being used as primers or as templates to be copied to generate false positive signals in non-specific reactions.

In an embodiment, the specific and/or quantitative detection of the nucleic acid sequence of interest comprises: (a) annealing amplification primers to strands of the target molecule comprising the nucleic acid sequence of interest; (b) amplifying the two strands of the target molecule between the first and second amplification primer sites in the presence of the polymerase (c) hybridizing the probe to a strand of the target molecule to form a probe:target duplex; (d) detecting a florescence emission after cleavage of a label off the probe using the 3′-5′ exonuclease activity of the polymerase; (e) extending the two primers using the target molecules as the templates; (f) repeating the above steps to amplify and detect the target nucleic acid sequence.

In an embodiment, a 3′ end of a primer of the primer set comprises a molecular moiety, wherein the molecular moiety is non-complementary to a target nucleic acid sequence of interest. A 3′ end of the primer set comprises a molecular moiety, wherein the molecular moiety is non-complementary to a target nucleic acid sequence of interest. The molecular moiety is non-complementary to each other.

In an embodiment, the molecular moiety is configured to be cleaved by the 3′-5′ exonuclease activity of the DNA polymerase prior to extension of a primer using the polymerase.

In an embodiment, the molecular moiety comprises a nucleotide and/or a nucleotide analogue selected from a group comprising an inosine, a uracil-containing nucleotide, an iso-deoxycytosine (iso-dC), an iso-deoxyguanosine (iso-dG), a diaminopurine, 2,4-difluorotoluene, 4-methylbenzimidazole, a size-expanded adenine (xA), a size-expanded guanine (xG), a size-expanded cytosine (xC), a size-expanded thymine (xT), 2-((2R,4R,5R)-tetrahydro-4-hydroxy-5-(hydroxymethyl) furan-2-yl)-6-methylisoquinoline-1(2H)-thione (d5SICS), 1,4-Anhydro-2-deoxy-1-C-(3-methoxy2-naphthalenyl)-(1R)-D-erythro-pentitol (dNaM), an abasic nucleotide, an acyclo nucleotide, a labeled nucleotide and/or combination thereof.

In an embodiment, the target molecule is generated by reverse transcription. The amplification reaction is conducted with a non-isolated nucleic acid sample. The amplification reaction performed using a thermal cycler or an isothermal device for convection-based heating.

An embodiment relates to a quantitative selective wobble (SW)method for nucleic acid amplification. A method comprising: (a) assembling a reaction mixture comprising: (i) a target molecule comprising a nucleic acid sequence of interest; (ii) a set of oligonucleotides comprising: (1) a 1st selective wobble (SW) primer comprising a 1st SW site; (2) a 2nd SW primer comprising a 2nd SW site; (3) at least a third primer; (4) a probe comprising: (i) an attenuating site, (ii) a first label in a non 3′ site and (iii) a second label at the 3′ end; (iv) a polymerase with 3′-5′ exonuclease activity; (b) conducting an amplification reaction of the target molecule comprising the nucleic acid sequence of interest using the reaction mixture; (c) detecting and amplifying the target molecule comprising the nucleic acid sequence of interest or variants thereof present in the target molecule. The SW sites are configured to enable non-disrupted nested amplification and quantification of the target molecule comprising the nucleic acid sequence of interest.

In an embodiment, a SW site comprises a nucleic acid sequence at least 1 to 10 nucleotides non-complementary to the target molecule comprising the nucleic acid sequence of interest.

In an embodiment, the primers comprising the 1st SW primer and the 2nd SW primer are configured to be either a forward SW primer set or a reverse SW primer set.

In an embodiment, the third primer is configured to be either a reverse primer or a forward primer. The third primer comprises a SW site optionally. In further embodiment, the third primer is used together with the SW primer set for nucleic acid amplification.

In an embodiment, the attenuating site of the probe further comprises a structure comprising a natural nucleotide, a non-natural nucleotide, an abasic site, a spacer, a fluorescent label-modified nucleotide, an atypical nucleotide in DNA sequence comprised of deoxyuridine, a chemically synthesized nucleotide or combination thereof. The attenuating site is located between the center of the probe and the second label. It is configured to prevent the probe molecules from being used as primers or templates to be copied to generate false positive signals in non-specific reactions.

In an embodiment, the 1st SW primer further comprises: (i) a 5′ anchor region, (ii) a first 5′ recognition region, (iii) a 3′ extension region, (iv) the 1st SW site between the 5′ recognition region and the 3′ extension region.

In an embodiment, the 2nd SW primer further comprises: (i) a second 5′ recognition region, (ii) a 3′ recognition region, (iii) the 2nd SW site being close to a central region.

In an embodiment, nested amplification of the target molecule comprising the nucleic acid sequence of interest comprises a SW method comprising: (i) extending the 1st SW primer using the polymerase to generate a mutated strand of the target molecule; (ii) generating a mutated complementary strand from the mutated strand using the third primer; (iii) amplifying the mutated complementary strand and the mutated strand using the 2nd SW primer and the third primer. The 2nd SW primer is configured to have perfect match with the mutated complementary strand.

In an embodiment, the second label at the 3′ end label of the probe is cleavable effectively using the 3′-5′ exonuclease activity of the polymerase when the probe is hybridized to its target to form a double-stranded structure. The first label and the second label of the probe comprises a fluorescent dye-quencher pair or similar thereof.

In an embodiment, a 3′ end of the 1st SW primer and/or 2nd primer comprises a molecular moiety. The molecular moiety is non-complementary to the target nucleic acid sequence of interest.

In an embodiment, the molecular moiety is configured to be cleaved prior to extension of the 1st SW primer and/or 2nd primer using the polymerase.

In an embodiment, the molecular moiety comprises a nucleotide and/or a nucleotide analogue selected from a group comprising an inosine, a uracil-containing nucleotide, an iso-deoxycytosine (iso-dC), an iso-deoxyguanosine (iso-dG), a diaminopurine, 2,4-difluorotoluene, 4-methylbenzimidazole, a size-expanded adenine (xA), a size-expanded guanine (xG), a size-expanded cytosine (xC), a size-expanded thymine (xT), 2-((2R,4R,5R)-tetrahydro-4-hydroxy-5-(hydroxymethyl) furan-2-yl)-6-methylisoquinoline-1(2H)-thione (d5SICS), 1,4-Anhydro-2-deoxy-1-C-(3-methoxy2-naphthalenyl)-(1R)-D-erythro-pentitol (dNaM), an abasic nucleotide, an acyclo nucleotide, a labeled nucleotide and/or combination thereof.

In an embodiment, the 1st SW primer further comprises an attenuating site of at least 1 to 10 units. The attenuating site comprises at least 1 to 10 units and wherein the polymerase during the extension or amplification reaction does not pass through the attenuating site of the 1st SW primer.

In an embodiment, the attenuating site further comprises a structure comprising a natural nucleotide, a non-natural nucleotide, an abasic site, a spacer, a fluorescent label-modified nucleotide, an atypical nucleotide comprised of deoxyuridine, a chemically synthesized nucleotide or combination thereof.

In an embodiment, the target molecule is generated by a reverse transcription. The amplification reaction is conducted with a non-isolated nucleic acid sample. The amplification reaction performed using a thermal cycler or an isothermal device for convection-based heating.

An embodiment relates to a composition configured to detect and quantify target molecules comprising a nucleic acid sequence of interest or variants thereof. A composition comprising: a primer set comprises a pair of amplification primers; a probe comprising: (i) an attenuating site, (ii) a first label in a non 3′ site and (iii) a second label at the 3′ end; a polymerase with a 3′-5′ exonuclease activity. The composition is configured to detect and quantify target nucleic acid molecules of interest or variants thereof by a non-disrupted nested nucleic acid amplification.

In an embodiment, the second label at the 3′ end of the probe is cleavable effectively using the 3′-5′ exonuclease activity of the polymerase.

In an embodiment, the first label and the second label comprise fluorescent dye-quencher pair or similar thereof.

In an embodiment, a 3′ end of the primer set comprises a molecular moiety, wherein the molecular moiety is non-complementary to a target molecule sequence of interest.

In an embodiment, the attenuating site comprises at least 1 to 10 units, selected from the group of a natural nucleotide, a non-natural nucleotide, an abasic site, a spacer, a fluorescent label-modified nucleotide, an atypical nucleotide comprised of deoxyuridine, a chemically synthesized nucleotide or combination thereof. The attenuating site is located between the center of the probe and the second label. It is configured to prevent the probe molecules from being used as primers or templates to be copied to generate false positive signals in non-specific reactions.

An embodiment relates to a composition configured to detect and/or quantify of target molecules comprising a nucleic acid sequence of interest or variants thereof by non-disrupted nested nucleic acid amplification. A composition comprising: a set of oligo nucleotides comprising: (a) a 1st SW primer comprising a 1st SW site; (b) a 2nd SW primer comprising a 2nd SW site; (c) at least a third primer; a probe comprising: (i) an attenuating site, (ii) a first label in a non 3′ site and (iii) a second label at the 3′ end; a polymerase with 3′-5′ exonuclease activity. The composition is configured to detect and/or quantify of target molecules comprising a nucleic acid sequence of interest or variants thereof by non-disrupted nested nucleic acid amplification.

In an embodiment, the 1st SW site and the 2nd SW site comprise a nucleic acid sequence at least 1 to 10 nucleotides non-complementary to the target molecule comprising the nucleic acid sequence of interest. The third primer comprises a SW site optionally.

In an embodiment, the 1st SW primer further comprises: (i) a 5′ anchor region, (ii) a first 5′ recognition region, (iii) a 3′ extension region, and (iv) a 1st SW site that is in between the 5′ recognition region and the 3′ extension region.

In an embodiment, the 2nd SW primer further comprises: (i) a second 5′ recognition region, (ii) a 3′ recognition region, (iii) the 2nd SW site being close to a central region.

In an embodiment, the first label and the second label of the probe comprise a fluorescent dye-quencher pair or similar thereof.

In an embodiment, a 3′ end of the 1st SW primer and/or 2nd primer comprises a molecular moiety. The molecular moiety is non-complementary to the target molecule comprising the nucleic acid sequence of interest.

In an embodiment, the 1st SW primer further comprises an attenuating site of at least 1 to 10 units, selected from the group of a natural nucleotide, a non-natural nucleotide, an abasic site, a spacer, a fluorescent label-modified nucleotide, an atypical nucleotide comprised of deoxyuridine, a chemically synthesized nucleotide or combination thereof.

An embodiment relates to a kit configured to detect and quantify target molecules comprising a nucleic acid sequence of interest or variants thereof. A kit comprising: a primer set comprises a pair of amplification primers; a probe comprising: (i) an attenuating site, (ii) a first label in a non 3′ site and (iii) a second label at the 3′ end; a polymerase with a 3′-5′ exonuclease activity. The kit is configured to detect and quantify target nucleic acid molecules of interest or variants thereof by a non-disrupted nested nucleic acid amplification.

In an embodiment, the second label at the 3′ end of the probe is cleavable effectively using the 3′-5′ exonuclease activity of the polymerase.

In an embodiment, the first label and the second label comprise fluorescent dye-quencher pair or similar thereof.

In an embodiment, a 3′ end of the primer set comprises a molecular moiety. The molecular moiety is non-complementary to a target molecule sequence of interest.

In an embodiment, the attenuating site comprises at least 1 to 10 units, selected from the group of a natural nucleotide, a non-natural nucleotide, an abasic site, a spacer, a fluorescent label-modified nucleotide, an atypical nucleotide comprised of deoxyuridine, a chemically synthesized nucleotide or combination thereof. The attenuating site is located between the center of the probe and the second label. It is configured to prevent the probe molecules from being used as primers or templates to be copied to generate false positive signals in non-specific reactions.

An embodiment relates to a kit is configured to detect and/or quantify of target molecules comprising a nucleic acid sequence of interest or variants thereof by non-disrupted nested nucleic acid amplification. A kit comprising: a set of oligo nucleotides comprising: (a) a 1st SW primer comprising a 1st SW site; (b) a 2nd SW primer comprising a 2nd SW site; (c) at least a third primer; a probe comprising: (i) an attenuating site, (ii) a first label in a non 3′ site and (iii) a second label at the 3′ end; a polymerase with 3′-5′ exonuclease activity. The kit is configured to detect and/or quantify of target molecules comprising a nucleic acid sequence of interest or variants thereof by non-disrupted nested nucleic acid amplification.

In an embodiment, the 1st SW site and the 2nd SW site comprise a nucleic acid sequence at least 1 to 10 nucleotides non-complementary to the target molecule comprising the nucleic acid sequence of interest. The third primer comprises a SW site optionally.

In an embodiment, the 1st SW primer further comprises: (i) a 5′anchor region, (ii) a first 5′ recognition region, (iii) a 3′ extension region, and (iv) a 1st SW site that is in between the 5′ recognition region and the 3′ extension region.

In an embodiment, the 2nd SW primer further comprises: (i) a second 5′ recognition region, (ii) a 3′ recognition region, (iii) the 2nd SW site being close to a central region.

In an embodiment, the first label and the second label of the probe comprise a fluorescent dye-quencher pair or similar thereof.

In an embodiment, a 3′ end of the 1st SW primer and/or 2nd primer comprises a molecular moiety, wherein the molecular moiety is non-complementary to the target molecule comprising the nucleic acid sequence of interest.

In an embodiment, the 1st SW primer further comprises an attenuating site of at least 1 to 10 units, selected from the group of a natural nucleotide, a non-natural nucleotide, an abasic site, a spacer, a fluorescent label-modified nucleotide, an atypical nucleotide comprised of deoxyuridine, a chemically synthesized nucleotide or combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1A-1C: shows primer designs and relative positions of primers and template.

FIG. 2: schematic drawing of an exemplary SW method, a single tube nested PCR process.

FIG. 3A-3B: shows first selective wobble primer FIG. 3A: represents 1st SW primer and 2nd SW primer. FIG. 3B: represents 2nd SW primer with 3′end molecular moiety (non-complementary ending moiety).

FIG. 4: schematic drawing of SW method with 3′ end molecular moiety (non-complementary ending moiety).

FIG. 5A-5B: shows 1st SW primer design and SW method. FIG. 5A: represents 1st SW primer with attenuating site. FIG. 5B: schematic drawing of SW method with 1st SW primer comprising attenuating site.

FIG. 6: shows a second selective wobble probe primer (2^{nd} SW probe primer) design and SW method. represents 2^{nd} SW probe primer with 5′ end 1^{st} label and 3′end 2^{nd} label. Schematic drawing of quantitative SW method with SW probe primer.

FIG. 7: schematic drawing SW method for nucleic acid amplification using an outer primer.

FIG. 8: schematic probe sequence to SARS-COV-2 N gene. (FIG. 8A) and schematic drawing quantitative PCR for target nucleic acid detection using probe with attenuating site (FIG. 8B).

FIG. 9: schematic drawing of probe hybridization to the target molecule and cleavage of the 2nd label at the 3′ end of the probe using the 3′-5′ exonuclease activity of the polymerase.

FIG. 10: schematic drawing of quantitative PCR for target nucleic acid detection using probe with attenuating site.

FIG. 11A-11B: shows primer design (FIG. 11A), and photograph of gel (FIG. 11B), showing the results of single tube nested PCR (SW method) detection of HBV DNA.

FIG. 12A-12B: shows target nucleic acid sequence, primer design (FIG. 12A) and amplification plot (FIG. 12B) for single tube quantitative-nested PCR (quantitative SW method) detection of SARS-COV-2 N gene.

FIG. 13A-13B: shows target nucleic acid sequence, primers, probe (FIG. 13A) and amplification plot (FIG. 13B) for quantitative PCR (quantitative method) detection of SARS-COV-2 N gene.

### DETAILED DESCRIPTION OF THE PREFERRED EMBODIMENT

### Definitions and General Techniques

For simplicity and clarity of illustration, the drawing figures illustrate the general manner of descriptions and techniques.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, kit, composition, article, device, or apparatus that comprises a list of elements is not necessarily limited to those elements, but may include other elements not expressly listed or inherent to such process, method, system, kit, composition, article, device, or apparatus.

The terms “left,” “right,” “front,” “back,” “top,” “bottom,” “over,” “under,” “forward”, “reverse”, and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

The term “mutation” refers to an alteration in a polynucleotide sequence. A polynucleotide sequence in which a mutation has occurred is called a “mutant”. Mutation may be introduced or occur to one or both strands of a double-stranded polynucleotide molecule. The strand of a double-stranded polynucleotide in which a mutation has occurred is referred to as a “mutated strand”. Generally, when the mutated strand is copied or replicated, the complementary strand will be also mutated in the corresponding site.

The term “selective wobble (SW) primer” refers to a polynucleotide primer used in an extension or amplification reaction, wherein mutations are selectively introduced so that the primer matches incompletely with the hybridization site of a target sequence (e.g., a primer site in a target DNA molecule). The region containing the mismatched nucleotides in the selective wobble (SW) primer is referred to as a “SW site” or “site” with respect to the target sequence (e.g., the sequence of a target DNA molecule). A SW primer is designed or configured so that it can be extended by DNA polymerase under proper conditions. Thus, during the extension or amplification reaction, the mismatched nucleotides of a SW primer are incorporated into the extended or amplified products thereby resulting in the synthesis of mutated complementary strands of the template. Thus, selective wobble (SW) primers are used to prime the synthesis of mutated target sequences. The mutated complementary strands can then be copied or amplified by another primer that hybridizes with the mutated complementary stand to generate a mutated template strand.

The term “mutated complementary strand” of the template refers to the product after introducing a SW site into a complementary strand synthesized during an extension or amplification reaction using the target template strand. SW sites are preferably introduced into a double-stranded DNA molecule through a selective wobble (SW) primer in an amplification reaction. During the amplification reaction, multiple copies of the complementary strand of the template are synthesized by hybridizing the selective wobble (SW) primer to the template strand and extending the hybridized primer using the target strand as a template.

The term “complementary” is used herein to refer to the broad concept of sequence complementarity between regions of two polynucleotide strands or between two regions of the same polynucleotide strand. It is known that an adenine (A) residue of a first polynucleotide region is capable of forming specific hydrogen bonds (“base pairing”) with a thymine (T) or a Uracil (U) residue of a second polynucleotide region which is antiparallel to the first region. Similarly, it is known that a cytosine (C) residue of a first polynucleotide strand is capable of base pairing with a residue of a second polynucleotide strand which is antiparallel to the first strand if the residue is guanine (G). A first region of a polynucleotide is complementary to a second region of the same or a different polynucleotide if, when the two regions are arranged in an antiparallel fashion, at least one nucleotide residue of the first region is capable of base pairing with a residue of the second region. A first polynucleotide that is 100% complementary to a second polynucleotide forms base pair at every nucleotide position. A first polynucleotide that is not 100% complementary (e.g., 90%, or 80% or 70% complementary) contains mismatched nucleotides at one or more nucleotide positions.

The term “DNA” is used herein to refer to Deoxyribonucleic acid (DNA) is an organic chemical of complex molecular structure that is found in all prokaryotic and eukaryotic cells and in many viruses. DNA codes genetic information for the transmission of inherited traits. DNA in prokaryotic and eukaryotic cells is composed of two polynucleotide chains that coil around each other to form a double helix. The nucleotides of DNA consist of a deoxyribose sugar molecule to which is attached a phosphate group and one of four nitrogenous bases: two purines (A for adenine and G for guanine) and two pyrimidines (C for cytosine and T for thymine).

The term “RNA” is used herein to refer to Ribonucleic acid (RNA), complex compound of high molecular weight that functions in cellular protein synthesis and replaces DNA as a carrier of genetic codes in some viruses. RNA consists of ribonucleotides (nitrogenous bases appended to a ribose sugar) attached by phosphodiester bonds, forming strands of varying lengths. The nitrogenous bases in RNA are adenine (A), guanine (G), cytosine (C), and uracil (U), which replaces thymine in DNA.

The term “polymerase chain reaction (PCR)” is used herein to refer to a method of amplifying a target sequence of nucleic acid, which involves repeated cycles of DNA replication, wherein 1) strands of DNA are denatured to form single-strand templates (the denaturing step); 2) the templates are treated with oligonucleotide primers (the annealing step); 3) a polymerase enzyme is used to extend the primers to produce replicated double-stranded DNA molecules (the extension step); and 4) the replicated DNA molecules then serve as templates for additional rounds of replication (repeating the above mentioned 3 steps). These steps can be carried out by synchronized thermal cycling or by un-synchronized convective thermal cycling.

The term “amplification” or “amplifying” is used herein to refer to any in vitro process for exponentially or linearly increasing the number of target molecules of a polynucleotide sequence or sequences. Nucleic acid amplification results in the incorporation of nucleotides, ribonucleotides or deoxyribonucleotides, into elongating strands primed by primers to form DNA or RNA polynucleotides complementary to template nucleic acid molecules. As used herein, one amplification reaction may consist of many rounds of primer extension. For example, one PCR reaction may consist of several cycles of denaturation, annealing and extension ranging from about 5 cycles to 1000 cycles, or more. Such thermal cycling can be synchronized by a thermocycler or unsynchronized with a convective thermal device (isothermal device). In a DNA amplification in a convective thermal device (isothermal device), the molecules undergoing cycling of denaturation, annealing and extension are not synchronized.

The term “amplicon” is used herein to refer to a piece of DNA or RNA that is the source and/or product of amplification or replication events. It can be formed artificially, using various methods including the polymerase chain reactions (PCR) or naturally through gene duplication.

The term “nucleic acid” or “nucleic acid molecule” is used herein to refer to a polymeric form of nucleotide monophosphates of any length. Biochemically a nucleic acid molecule is synthesized from deoxyribonucleoside triphosphates (dNTPs), ribonucleoside triphosphates (rNTPs), their analogues thereof and/or combinations thereof. Nucleic acids can also be made chemically using methods known in the art. Nucleic acids may have any three-dimensional structure, and may perform any function, known or unknown. Non-limiting examples of nucleic acids include deoxyribonucleic acid (DNA), ribonucleic acid (RNA), a peptide nucleic acid (PNA), a locked nucleic acid (LNA), fluorinated nucleic acids (FNA), bridged nucleic acids (BNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides, dye-modified nucleotides, quencher-labeled nucleotides and nucleotide analogues. If present, modifications to the nucleotide structure may be made before or after assembly of the nucleic acid. The sequence of nucleotides of a nucleic acid may be interrupted by non-nucleotide components such as for example, a linker, a basic structure, or a spacer species.

The term “nucleotide” is used herein to refer to a base-sugar-phosphate combination. Nucleotides are the monomeric units of nucleic acid polymers, e.g. DNA or RNA. The term includes ribonucleoside triphosphates, such as rATP, rCTP, rGTP, or rUTP, collectively called rNTPs or NTPs and deoxy-ribonucleoside triphosphates, such as dATP, dCTP, dUTP, dGTP, or dTTP collectively called dNTPs. A “nucleoside” is a base-sugar combination, e.g. a nucleotide lacking phosphate. It is recognized in the art that there is certain interchangeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxy-uridylate, i.e. dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

The term “nucleic acid sequence” is used herein to refer to a series of contiguous nucleotides or bases arranged in certain or given order. The terms of nucleotides, bases or nucleobases are used herein as the basic units of a nucleic acid sequence and thus can interexchange. A nucleic acid sequence can also mean a nucleic acid molecule (such as DNA, cDNA or RNA) that contains a given nucleotide sequence or its complementary sequence. Thus, the term “a target molecule”, or “a target nucleic acid molecule”, or “a target nucleic acid sequence”, or “nucleic acid sequence of interest”, or “target nucleic acid” refers to a nucleic acid sequence or molecule that is to be detected or amplified. The term “target nucleic acid sequence”, “nucleic acid sequence of interest”, “target nucleic acid molecule”, “target molecule” and “target nucleic acid” are used interchangeably. One specific target nucleic acid sequence is a segment, region, or fragment of a nucleic acid molecule that can hybridize with a primer. Depending on the context, the terms of nucleic acid, sequence, target, molecules, template, and their combinations, such as nucleic acid sequence, target sequence, target nucleic acid sequence, nucleic acid molecule, are used interchangeably. In an embodiment, the nucleic acid sequence of interest is a sequence derived from SARS-CoV-2 genome RNA sequence. The derived sequence can be a fragment, a region of the genomic RNA sequence. The derived sequence can also be a cDNA sequence that is reverse-transcribed from the genomic RNA sequence. The reverse transcription (RT) can take place in the same tube prior to the PCR reaction (e.g., including nested PCR, probed-PCR, quantitative PCR, or combination thereof); thus a RT-PCR can be performed in the same tube, using the same reaction mixture for the detection of a pathogen, such as the SARS-CoV-2 virus that is the causative agent for COVID-19.

The term “anneal or annealing” is used herein to refer to a binding of one nucleic acid molecule (e.g., a primer) with another nucleic acid molecule (e.g., a template nucleic acid molecule) via complementarity between the nucleic acid molecules following the conventional base-paring rules, where A pairs with T or U, and C pairs with G.

The term “denaturing” and “denaturation” is used herein to refer the full or partial unwinding of the helical structure of a double-stranded nucleic acid molecule, and in some embodiments the unwinding of the secondary structure of a single stranded nucleic acid.

The term “reaction mixture” or “amplification reaction mixture” is a composition comprising one or more reagents necessary to complete a primer extension reaction, a reverse transcription and/or nucleic acid amplification, with non-limiting examples of such reagents that include one or more primers having specificity for a target nucleic acid, such as random primers for non-specific reverse transcriptions, a DNA polymerase, suitable buffers, co-factors (e.g., divalent and monovalent cations), nucleotides (e.g., deoxyribonucleoside triphosphates (dNTPs)), and any other enzymes, such as a reverse transcriptase. In some embodiments, a reaction mixture can also comprise one or more detectable species, for example, a florescent dye and quencher.

The term “amplification reaction” is used herein to refer to any in vitro means for multiplying the molecules of a target sequence of nucleic acid.

The term “terminating” is used herein refer to causing a treatment to stop. The term includes both permanent and temporary or conditional stoppages. For example, if a treatment were enzymatic, a permanent stoppage might be heat denaturation of the molecule or molecules that catalyzes the enzymatic treatment. A conditional stoppage might be, for example, incubation at a temperature outside the active range of the molecule or molecules that catalyzes the enzymatic treatment but at which temperature the molecules are not made permanently inactive. Both types of termination are intended to fall within the scope of this term.

The term “oligonucleotide” is used herein to refer to various lengths of single-stranded nucleic acid molecules (RNA or DNA). The term is used collectively and interchangeably with other terms of the art such as “polynucleotide”, ‘primer” and “probe.” Note that although oligonucleotide, polynucleotide, primer and probe are distinct terms of art, there is no exact dividing line between them. These terms are used interchangeably herein.

The term “primer” or “amplification primer” is used herein to refer to a single-stranded oligonucleotide or a single-stranded polynucleotide that is capable of hybridizing to a template molecule and initiating the extension by covalent addition of nucleotide monomers, for example, during an amplification reaction. Nucleic acid amplification often is based on nucleic acid synthesis by a nucleic acid polymerase. Many such polymerases require the presence of a primer that can be extended to initiate such nucleic acid synthesis.

The term “3∝” is used herein to refer to a downstream direction, a region or a position in a polynucleotide or oligonucleotide 3′ (downstream) from another region or position in the same polynucleotide or oligonucleotide.

The term “5′” is used herein to refer to an upstream direction, a region or a position in a polynucleotide or oligonucleotide 5′ (upstream) from another region or position in the same polynucleotide or oligonucleotide.

The phrase “oligonucleotide-dependent amplification” is used herein to refer to amplification using an oligonucleotide, or polynucleotide, or probe or primer to amplify a nucleic acid molecule. An oligonucleotide-dependent amplification is any amplification that requires the presence of one or more oligonucleotides or polynucleotides or probes or primers that are two or more mononucleotide subunits in length and end up as part of the newly formed, amplified nucleic acid molecules. The phrase “template-dependent amplification” is used herein to refer to nucleic acid amplification involving copying or replicating a nucleic acid template molecule. Typically, template-dependent amplification also involves primers.

The phrase “thermostable polymerase” is used herein to refer to an enzyme that is relatively stable to heat and is capable of catalyzing the formation of DNA or RNA from an existing nucleic acid template. One example of a thermostable polymerase is a thermostable DNA polymerase, which is relatively stable to heat and is capable of catalyzing the polymerization of nucleoside triphosphates to form primer extension products that are complementary to one of the nucleic acid strands of the target sequence. The enzyme initiates synthesis at the 3′ end of the primer and proceeds in the direction toward the 5′ end of the template until synthesis terminates. Based on their structures and properties, DNA polymerases can be classified into different families. Family A DNA polymerase includes Taq DNA polymerase that has a 5′-3′ exonuclease activity, the thermostable DNA polymerase most commonly used in PCR. Family B DNA polymerases include Pfu DNA polymerase. Family B polymerases are highly accurate in their function and perform proofreading of newly synthesized DNA by 3′-5′ exonuclease activity in order to correct any errors that occur during DNA replication. These as well as other thermal DNA polymerases from various commercial vendors can be used for nucleic acid amplification or PCR. The 3′-5′ exonuclease activity of a DNA polymerase can sequentially cleave nucleotide one by one from the 3′ end of a nucleic acid strand. The preferred substrates are mismatched nucleotides in double-stranded DNA molecules. Thus, non-complementary nucleotides, nucleotide analogues or molecular moieties are cleaved efficiently when corresponding primers or probes are hybridized or annealed to form double-stranded structures.

The term “primer extension reaction” is used herein to refer to a binding (e.g., “annealing”) of a primer to a strand of nucleic acid, followed by incorporation of nucleotides to the primer (e.g., “extension of” or “extending” the primer) often at its 3′ end, using the strand of nucleic acid as a template. A primer extension reaction may be completed with the aid of an enzyme, such as, for example a polymerase.

The term “melting temperature (Tm)” is used herein to refer to a temperature at which two single-stranded nucleic acid molecules that are hybridized and form a double-stranded molecule dissociate from each other. The melting temperature can refer to a temperature at which about 5%, 10%, 15%, 20%, 25%, 30%, 35%, 40%, 45%, 50%, 55%, 60%, 65%, 70%, 75%, 80%, 85%, 90%, 95%, 99% or more identical nucleic acid strands of a population of identical double-stranded nucleic acid molecules dissociate from their respective complement strands. For example, the melting temperature of a primer or molecular moiety may refer to the temperature at which about half (50%) of the molecules of the primer or molecular moiety in a population of identical primers or molecular moieties hybridized to a nucleic acid molecule dissociate from their complementary sequence on their respective nucleic acid molecules. A melting temperature of a nucleic acid molecule can be calculated based on the sequence of the nucleic acid molecule via any suitable calculation method.

The term “thermal cycler” is used herein to refer to an instrument for use in a nucleic acid amplification reaction comprising multiple thermal cycles for alternately heating or cooling samples. The term “convective thermal device” is used herein to refer to an instrument for use in temperature control, resulting in liquid convection due to density gradients, in which the lower portion of a test tube containing a reaction mixture is set to a temperature higher (lower liquid density) than that of the upper portion of the same test tube (higher liquid density).

The term “sample” is used herein to refer to any nucleic-acid-containing specimen to be tested. The sample can be any biological material that contains nucleic acid molecules suitable for practicing the methods of the invention.

The term “purified” is used herein to indicate that a molecule of interest has been separated from some or all other surrounding molecules and/or materials. “Purified” is thus a relative term, which is based on a change of the desired molecule in close proximity to other molecules, i.e. in a free state. Enzymes, for example, which adhere to, attach to, bind to (covalently or non-covalently), and/or associate with other biological or non-biological material after cell lysis are considered to be purified when at least some cellular debris, proteins and/or carbohydrates are removed by washing. These same enzymes are purified again, when they are released from other materials using methods or compositions of the invention.

The term “isolated” is used herein to indicate that a molecule of interest has been separated from substantially all of the molecules and/or materials that it is associated with it in its natural state. Alternatively, isolated means when the molecule is set apart or free from other molecules (a step of nucleic acid isolation). To determine whether a biological molecule has been isolated, the concentration of materials such as water, salts, and buffer are not considered when determining whether a biological molecule has been “isolated.” Thus, a non-isolated nucleic acid sample is a sample that does not go through the step of nucleic acid isolation.

The term “template” is used herein to refer to a strand used by DNA polymerase or RNA polymerase to attach complementary bases during DNA replication or RNA transcription, respectively; either molecule moves down the strand in the 5′-3′ direction (or 3′-5′direction of the template), and at each subsequent base, it adds the complement of the current DNA base to the growing nucleic acid strand (which is thus created in the 5′-3′ direction).

The term “dNTP” refers to deoxyribonucleoside triphosphates. The purine bases (Pu) include adenine (A), guanine (G) and derivatives and analogues thereof. The pyrimidine bases (Py) include cytosine (C), thymine (T), uracil (U) and derivatives and analogues thereof. Examples of such derivatives or analogues, by way of illustration and not limitation, are those which are modified with a reporter group, biotinylated, amine modified, radio-labeled, alkylated, and the like and also include phosphorothioate, phosphite, nucleobase ring atom modified derivatives, and the like. The reporter group can be a fluorescent group such as fluorescein, a quencher, a chemiluminescent group such as luminol, a terbium chelator such as N-(hydroxyethyl) ethylenediaminetriacetic acid that is capable of detection by delayed fluorescence, and the like.

The term “quantitative PCR (qPCR)”, or “real-time PCR” is used herein to refer to a PCR-based technique that couples amplification of a target DNA sequence with quantification of the input concentration of that DNA species in the reaction. qPCR uses the logarithmic scale of DNA amplification to determine absolute or relative quantities of a known sequence in a sample. By using a fluorescent reporter in the reaction, it is possible to measure DNA amplification in the qPCR assay. In qPCR, DNA amplification is monitored at each cycle of PCR. When the DNA is in the exponential phase of amplification, the amount of fluorescence increases above the background. The point at which the fluorescence becomes measurable is called the threshold cycle (CT) or crossing point. By using multiple dilutions of a known amount of standard DNA, a standard curve can be generated of log concentration against CT. The input amount of DNA or cDNA or RNA in an unknown sample can then be calculated from its CT value in a PCR (for DNA as input template) or RT-PCR reaction (for RNA as input template). When a convective thermal device is used for nucleic acid amplification, “quantitative PCR” or quantification is measured by inflection points of fluorescent signal changes over time. When a convective thermal device is used for nucleic acid amplification, “real-time PCR” refers to real time data collection and real-time display.

The term “hybridization” is used herein to refer to an association of complementary strands of RNA or DNA to form a double-stranded molecule of DNA-DNA, DNA-RNA, or RNA-RNA.

The term “probe” is used herein to refer to a labeled polynucleotide or oligonucleotide sequence which is complementary to a polynucleotide or oligonucleotide sequence of a particular analyte and which hybridizes to said analyte. In this invention, an analyte is a target nucleic acid sequence of interest. A probe typically comprises one or more labels. A label is a tag or detectable moiety attached to the probe molecule. For example, a probe can have a quencher label or a fluorescent dye label.

The term “amplification plots” is used herein to refer to a plots are created when the fluorescent signal from each sample is plotted against cycle number; therefore, amplification plots represent the accumulation of product over the duration of the qPCR experiment. In a real qPCR assay, a positive reaction is detected by accumulation of a fluorescent signal. The Ct (cycle threshold) is defined as the number of cycles required for the fluorescent signal to cross the threshold (i.e. exceeding background level). The samples used to create the plots are a dilution series of the target DNA sequence. When a connective thermal device is used for unsynchronized nucleic acid amplification, the fluorescent signals can be monitored and displayed in real-time. The input template quantity is thus measured, instead of Ct, by time of inflection point that is inversely proportional to the amount of input nucleic acid template molecules.

The term “quencher” is used herein to refer to a molecule or molecular moiety that absorbs the fluorescence emission of reporter when in close vicinity (2 to 50 nucleotides apart). Commonly used quenchers include TAMRA (fluorescent), and non-fluorescent ones DABCYL and black hole quencher (BHQ) dyes. The quenchers are usually at one end of a dual-labeled probe. Quencher dye is also called acceptor. A quencher's efficiency increases as its absorption spectral overlaps the fluorescence emission profile of the reporter dye and as quencher absorption profile broadens (highest for BHQ).

The term “TaqMan probe” is used herein to refer to a probe structure where the 5′ label can be cleaved (hydrolysed) by the 5′-3′ exonuclease activity of Taq DNA polymerase. The probes are designed to increase the specificity of quantitative PCR. The TaqMan probe principle relies on the 5′-3′ exonuclease activity of Taq DNA polymerase to cleave a dual-labeled probe during hybridization to the complementary target sequence and fluorophore-based detection. As in other quantitative PCR methods, the resulting fluorescence signal permits quantitative measurements of the accumulation of the product during the exponential stages of the PCR; however, the TaqMan probe significantly increases the specificity of the detection. The 3′ end of a TaqMan probe is generally not cleavable by 3′-5′ exonuclease. When it cleaved near its 3′ end by endonuclease, the resulting oligonucleotide will serve as a primer for extension or as a template to be copied once is extended.

The term “hybridization probe” is used herein to refer to a fragment of DNA or RNA of variable length (usually 10-1000 bases long) which can be radioactively or fluorescently labeled. It can then be used in DNA or RNA samples to detect the presence of nucleotide substances (the RNA target) that are complementary to the sequence in the probe. The probe thereby hybridizes to single-stranded nucleic acid (DNA or RNA) whose base sequence allows probe-target base pairing due to complementarity between the probe and target.

The term “exonuclease activity” is used herein to refer to an enzyme activity that works by cleaving nucleotides one at a time from the end (exo) of a polynucleotide chain. A hydrolyzing reaction that breaks phosphodiester bonds at either the 3′ or the 5′ end occurs. Its close relative is the endonuclease activity. However, commonly used PCR enzyme Taq DNA polymerase has 5′-3′ exonuclease activity that is the fundamental feature of currently known qPCR methods.

The term “fluorescent dye” or “fluorescent label” is used herein to refer to a molecule or molecular moiety that absorbs a quantum of electromagnetic radiation at one wavelength, and emits one or more photons at a different, typically longer, wavelength in response thereto.

The term “virus” is used herein to refer to a sub-microscopic infectious agent that is unable to grow or reproduce outside a host cell. It is non-cellular but consisting of a core of DNA or RNA surrounded by a protein coat. A virus is a small parasite that cannot reproduce by itself. Once it infects a susceptible cell, however, a virus can direct the cell machinery to produce more virus particles.

An embodiment relates, in part, methods for the quantification and detection of target nucleic acid sequences by nested amplification. More specifically, the embodiments provide, in part, methods for single-tube quantitative nested PCR and real-time or quantitative PCR of a target nucleic acid sequence. At least three primers, which in most instances differ in nucleotide sequence, are used in amplification reactions. Typically, at least one of the primers will contain at least one SW site or a mismatched region. Further, in many instances, these SW sites, in combination with other regions present in the primers, will be used to alter the hybridization of the primers with the template nucleic acid molecules during an amplification reaction. For example, locations of SW sites present in the primers can be used, in conjunction with other regions of primers or conditions to alter the binding affinity of the primers to the template for the purposes of becoming involved in amplification reactions. In other words, to increase the possibility of hybridization of primers which are capable of functioning in amplification reactions in a time course fashion. A target molecule comprising a nucleic acid sequence of interest. The target molecule could be single-stranded or a double stranded nucleic acid sequence of DNA or RNA.

An embodiment provides a methods, compositions and kits that utilize probes with an attenuating site that cannot be extended by a polymerase activity and/or prevent from being copied during PCR. The attenuating site is located between the center of the probe and the second label. It is configured to prevent the probe molecules from being used as primers or templates to be copied to generate false positive signals in non-specific reactions. The probes with an attenuating site also comprise a first label in a non 3′ site and a second label at the 3′ end. A non 3′ site is a 5′ end position or an internal site that is 5′ upstream from the second label of the probe molecule. The second label at the 3′ end of the probe is cleavable effectively using the 3′-5′ exonuclease activity of the polymerase when the probe is hybridized to its target to form a double-stranded structure. The second label is 3′ downstream from the first label, separated by 1-50 nucleotides. Thus, the second label at the 3′ end can be a label in the 3′ direction relative to the first label; it can be in the terminal position or in an internal site near the 3′ terminus comprising 1-3 units. The second label can be cleaved effectively when the label-associated nucleotide and the terminal nucleotides are mismatched with its corresponding nucleotides in the template. Spacer C3 incorporated at the 3′-end of an oligo functions as an effective blocking agent against polymerase extension in PCR reactions. The 3′ end label of the probe is cleavable: effectively cleavable, meaning either cleaved directly from the polynucleotide it is being associated or together with the nucleotide it is being associated. A label can be a quencher or fluorescent dye. In a probe, one of the labels can be a fluorescent dye and the other can be a quencher. The quencher and the dye forms a fluorescent dye-quencher pair, in which the quencher is able to absorb the fluorescence emission of the dye when it is excited. In an embodiment, a C3 spacer is placed 5′ next to the second label in a probe molecule that has a non 3′ first label. When the probe is hybridized to its target molecule, the second label is cleaved by the 3′-5′ exonuclease activity of the DNA polymerase, resulting in a C3 blocked oligonucleotide and increased fluorescence emission.

An embodiment provides a reaction mixture that includes one or two forward primers, a probe, one or two-reverse primers, and a target molecule comprising a nucleic acid sequence of interest including a target region, such as a sequence derived from SARS-CoV-2 virus, a DNA polymerase with 3′-5′ exonuclease activity, a reverse transcriptase, random primers, a set of dNTPs and a buffer system. The primers with SW sites are also known as selective wobble (SW) primers. The SW primers could either be forward or reverse primers or both. The forward primers and the reverse primer are useful for amplifying the region of the template polynucleotide that includes the target region. The third primer is configured to be either a reverse primer or a forward primer, meaning in the opposite direction relative to the SW primers so that a PCR reaction can take place. The reaction mixture can additionally include an outer primer, where an outer primer is configured to amplify or enrich the templates nucleic acid molecules for the SW primers.

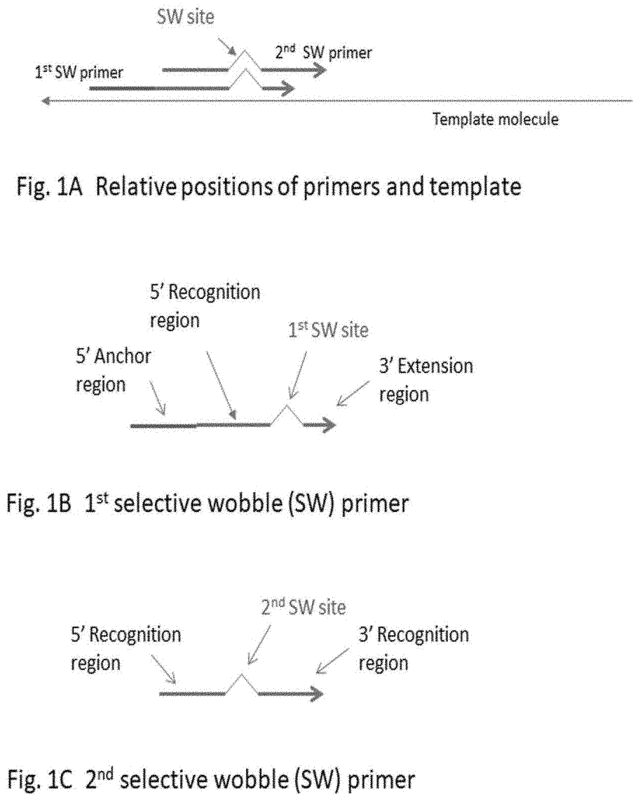

In one embodiment, a 1st SW primer comprises (i) a long 5′anchor region, (ii) a 5′ recognition region, (iii) a 3′ extension region, and (iv) a SW site wherein the SW site is in between the 5′ recognition region and the 3′ extension region (as illustrated in FIG. 1B). A 2nd SW primer comprises a 5′ recognition region, (ii) a long 3′recognition region, and (iii) a SW site wherein the second SW site is close to a central region (as illustrated in FIG. 1C). The 5′ recognition region of both the primers (1st SW primer and 2nd SW primer) is overlapping to each other and complementary to the target molecules. The 3′ recognition region of the 2nd SW primer is relatively longer than 3′ extension region of the 1st SW primer (as illustrated in FIG. 1A). The primers comprising the 1st SW primer and the 2nd SW primer are configured to be either a forward SW primer set or a reverse SW primer set. The third primer is configured to be either a reverse primer or a forward primer, the third primer comprises a SW site optionally.

An embodiment relates to a SW (selective wobble) site of a primer. Here the term “selective wobble” refers to a primer region that is consisted of 1-10 nucleotides not complementary to the target or template sequence. In some embodiments, forward primers used in the reaction mixture are complementary to a nucleic acid template except for at least 1-10 nucleotides long mismatched region. In some embodiments, reverse primers used in the reaction mixture are complementary to a nucleic acid template except for at least 1-10 nucleotides long mismatched region. The at least 1-10 nucleotides long mismatched region is called as SW site. The SW site is a nucleic acid sequence at least 1 to 10 nucleotides non-complementary to the template. These SW sites are non-complementary to a target nucleic acid sequence. Primers having these SW sites are called as selective wobble (SW) primers.

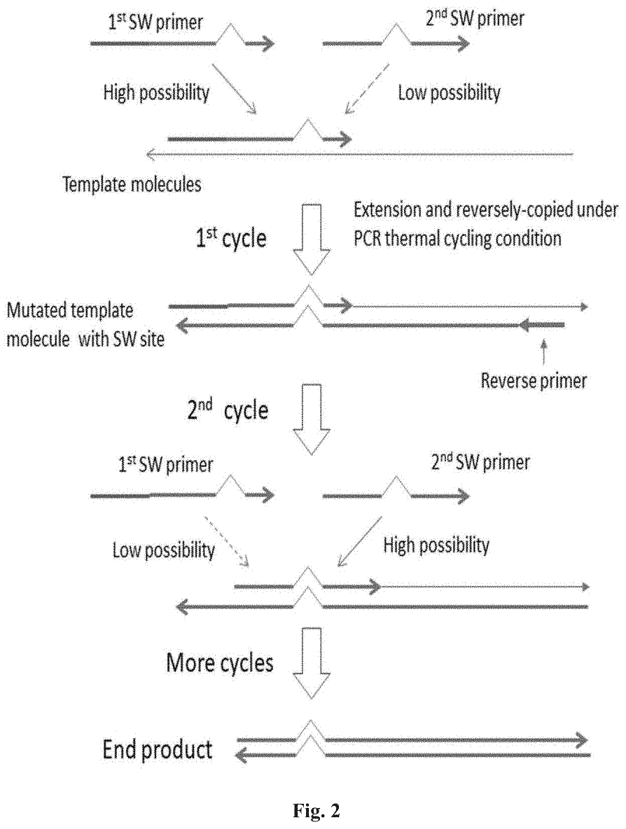

In one embodiment, a 1st SW primer has a SW site is close to or near to 3′ end of the 1st SW primer. In the method of non-disrupted nested amplification of the target molecule in a single tube nested PCR, in the beginning of the nested PCR or in the first cycle of single tube nested PCR (as illustrated in FIG. 2), the forward primer or 1st SW primer with a long 5′-anchor sequence anneals to the target template molecule at the specific site to form stable hybrid with the target template molecule. Extension of the 3′ end which is complementary to the target template molecule occurs in a primer extension reaction via the action of the polymerase to generate a mutated strand complementary to the target template molecule. The mutated strand generated in the first cycle of single-tube nested PCR comprises the SW site of the 1st SW primer. In the next cycle of the single-tube nested PCR, the mutated strand is copied using the reverse primer. The reverse primer extends and copies the SW site into the newly synthesized template molecule, creating a mutated complementary strand (or mutated template) which is complementary to the mutated strand generated by the 1st SW primer.

In one embodiment, the 1st SW primer having an anchor sequence (as illustrated in FIG. 1B). The anchor sequence is designed to form a stable hybrid with the template. The term “anchor sequence” used herein means a sequence which is positioned at the 5′ terminus of the 1st SW primer that is complementary with the target template molecule. The resulting mutated complementary strand (complementary to the template) is copied with another primer and copied strand becomes the target template sequence for the 2nd SW primer. The SW site of the 2nd SW primer is not complementary to the original input template molecule but is complementary to the mutated template derived from the mutated strand extended from the 1st SW primer. The length of the anchor sequence of the 1st SW primer (including a primer of a primer set) or oligonucleotide (including an oligonucleotide of an oligonucleotide set) described herein may vary depending upon the particular primer or oligonucleotide. In some embodiments, the length of the anchor sequence of a primer or oligonucleotide described herein may be from about 2 nucleotides to about 20 nucleotides long.

In one embodiment, the 1st SW primer has at least one primer extension region, onto which the primer is extended by a DNA polymerase. In some embodiments, the primer extension region is located at the 3′ end of the 1st SW primer downstream from the anchor region, recognition region and SW site. The length of the primer extension region of the 1st SW primer (including a primer of a primer set) or oligonucleotide (including an oligonucleotide of an oligonucleotide set) described herein may vary depending upon the particular primer or oligonucleotide. In some embodiments, the length of the primer extension region of a primer or oligonucleotide described herein may be from about 2 nucleotides to about 20 nucleotides long. During a primer extension reaction, a polymerase can generally add, in template-directed fashion, nucleotides to the 3′ end of a primer annealed to a single-stranded nucleic acid template molecule.

In one embodiment, the 2nd SW primer comprises the SW site close to central region of the 2nd SW primer. More preferably, the SW site in the 2nd SW primer is downstream of the 5′-recognition region. Due to SW site close to or near to the central region of the 2nd SW primer, the hybrid between a 2nd SW primer and an original input template nucleic acid molecule is far less stable than the hybrid between the 1st SW primer and the target template molecule. Therefore, the 2nd SW primer have less chance to be extended to form mutated strand of the target template molecule.

In one embodiment, the 2nd SW primer copies or amplifies the mutated complementary strand generated by the reverse primer. The mutated strand comprises the SW site. Due to the long 3′ recognition region of 2nd SW primer and the matched SW site with the mutated region, the 2nd SW primer has high possibility of annealing to the mutated complementary strand and form a stable hybrid. Because the 2nd SW primer is located relatively downstream from the 1st SW primer and generate shortened or truncated products, in the subsequent cycles of the single-tube nested PCR, the 2nd SW primer has high affinity for the mutated complementary strand than the 1st SW primer.

In one embodiment, the long 3′-recognition region of the 2nd SW primer and matched SW site with the mutated sequence enables the 2nd SW primer to stably hybridize with mutated complementary strand in comparison to the 1st SW primer. The 2nd SW primer become fully complementary to the mutated complementary strand. The amplification of the mutated complementary strand with the 2nd SW primer become more efficient. The 2nd SW primer are thus “dropped in” in the later cycles of the single-tube PCR by proceeding with a high possibility of annealing to the mutated template strand and 1st SW primer amplification is a “dropped out” by lower binding affinity for the mutated but truncated complementary strand. Thus, in the later cycles of the single-tube nested PCR “primer-switching” happens from 1st SW primer to the 2nd SW primer in the nested amplification of the target molecule.

In one embodiment, a probe comprising: (i) an attenuating site, (ii) a first label in a non 3′ site and (iii) a second label at the 3′ end. The attenuating site is located between the center of the probe and the second label. It is configured to prevent the probe molecules from being used as primers or templates to be copied to generate false positive signals in non-specific reactions. A non 3′ site is a 5′ end position or an internal site that is 5′ upstream from the second label of the probe molecule. The second label is 3′ downstream from the first label, separated by 1-50 nucleotides. Thus, the second label at the 3′ end can be a label in the 3′ direction relative to the first label; it can be in the terminal position or in an internal site near the 3′ terminus comprising 1-3 units.

In one embodiment, the method of the single-tube nested PCR comprises (as illustrated in FIG. 2): annealing of the 1st SW primer with the target template molecule to form a stable hybrid. Extension of the 3′ end which is complementary to the target template molecule occurs in a primer extension reaction via the action of the polymerase to generate a mutated strand of the target template molecule. The mutated stand generated in the first cycle of single-tube nested PCR comprising the SW site of the 1st SW primer. In the next cycle of the single-tube nested PCR, the mutated strand of the target template molecule is copied using the reverse primer. Extension with the reverse primer results in generation of the mutated complementary strand (template molecule with SW site). The 2nd SW primer copies or amplifies the mutated complementary strand with high affinity.

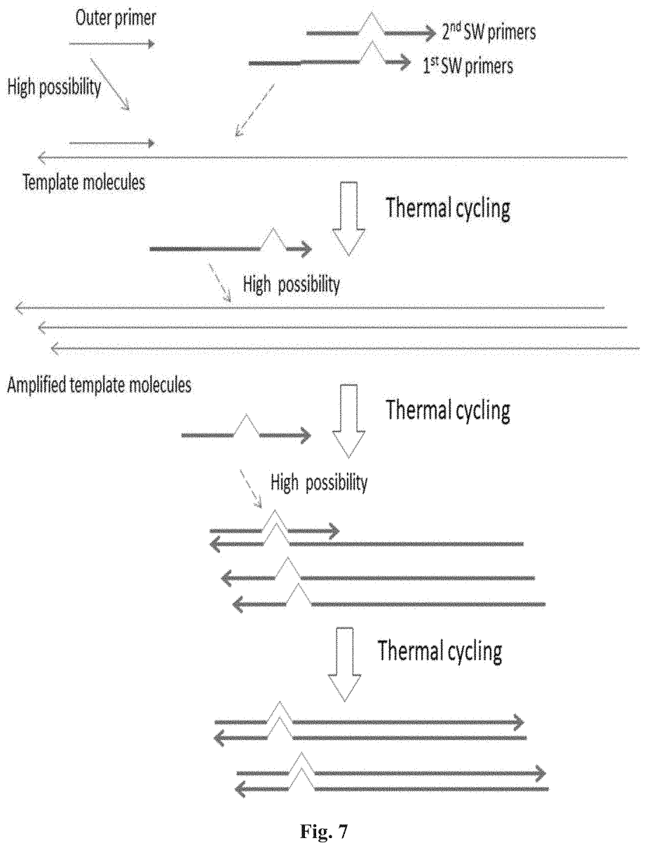

In one embodiment, forward primers can include an outer primer and the SW primers as the inner primers. The method therefore enables an initial amplification off the 1st SW primer to initially proceed efficiently as the dominant amplification reaction when more template molecules are generated when the outer primer is involved in initial amplification. However, since it is sought to conclude this amplification and to proceed with amplification off the 2nd SW primer. Together with the changes to the conditions, the ongoing unwanted amplification of the outer primers is minimised, and the amplification of the inner primers can proceed under conditions which facilitate efficient amplification. The outer primer is used to increase the detection sensitivity by provide more template molecules for forward primers (as illustrated in FIG. 7).

In one embodiment, the design and synthesis of primers suitable for use in the present invention would be well known to those of skill in the art. The Tm values of the primers and oligonucleotides would be largely determined by the annealing temperature which is desired for the various phases of the PCR. The subject primer may be of any suitable length which achieves the functional objective.

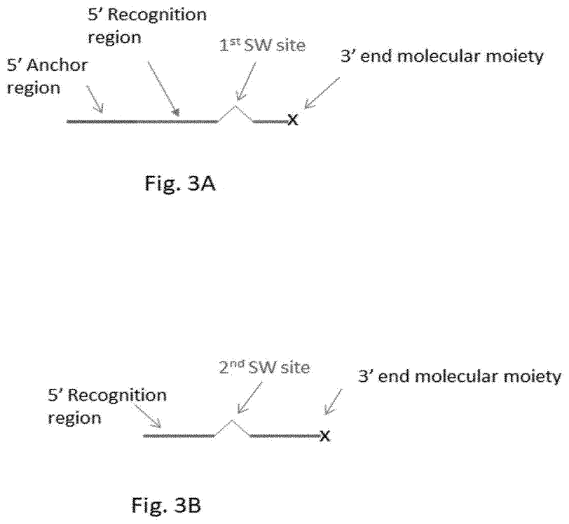

An embodiment relates to a primer (including a primer of a primer set) or oligonucleotide (including an oligonucleotide of an oligonucleotide set) described herein may comprise a molecular moiety at its 3′ end that is non-complementary and/or non-binding with respect to a target nucleic acid molecule (3′ non-complementary moiety, as illustrated in FIG. 3). A molecular moiety at the 3′ end has three functional features: (i) not extendable or not extended efficiently due to non-complementary, (ii) cleavable enzymatically to render the oligonucleotide extendable, (iii) if extended, not be copied due to non-complementary or strong polymerase binding. Example for strong polymerase binding to a nucleic acid unit is the archaebacterial polymerase binding to the nucleotide containing a uracil. Thus a primer with a 3′ molecular moiety must be removed in order for efficiently nucleic acid amplification.

In one embodiment, a molecular moiety of a primer or oligonucleotide described herein may be adapted to prevent the formation of a primer dimer by-product that comprises the primer (e.g., a forward primer) or an oligonucleotide dimer by-product that comprises the oligonucleotide. For example, the presence of a molecular moiety in a primer or oligonucleotide described herein may reduce the binding affinity (or prevent binding) of the primer or oligonucleotide for an additional primer or oligonucleotide. The presence of a molecular moiety may also reduce or eliminate the possibility that the primer or oligonucleotide can be extended in an amplification reaction, using, for example, another primer or oligonucleotide as a template. Upon removal of the molecular moiety by the 3′-5′ exonuclease activity of a DNA polymerase, the primer or oligonucleotide can then be extended. This can happen during a PCR reaction, in which the primer with a 3′ non-complementary moiety hybridizes a template molecule followed by the binding of a DNA polymerase. The DNA polymerase encounters the 3′ non-complementary moiety and cleave it using its 3′-5′ exonuclease activity before extending the primer. This can happen to one of or both the forward and reverse primer at the same time in the same amplification cycle or at different time in a convective thermal cycle. In the case of a primer set comprising a 1st SW primer and a 2nd SW primer each comprising a molecular moiety, one or both of the molecular moieties may be adapted to prevent the formation of a primer dimer molecular complex comprising the forward primer and/or reverse primer.

In one embodiment, the molecular moiety may be any suitable species. The molecular moiety may comprise one or more phosphodiester bonds. Non-limiting examples of molecular moieties include nucleotides, nucleic acids and non-nucleotide species (e.g., amino acids, peptides, proteins, carbohydrates, hydrocarbon chains (e.g., polyethylene glycol (PEG)), an n-phosphate moiety (where “n” is greater than or equal to 1, 2, 3, 4, 5, 6, 7, 8, 9, or 10), phosphodiester bond linked hetero-conjugates, dyes and organic-metal complexes). Moreover, a molecular moiety may also comprise individual subunits or species linked together (either continuously or discontinuously) via covalent bonds. Such individual species or subunits may be, for example, one or more individual nucleotides of a nucleic acid, one or more amino acids of a peptide or protein, or one or more sugars of a carbohydrate. For example, the length of a molecular moiety may be about 1 to 20, 1 to 15, 1 to 10 or 1 to 5 individual specie or subunits. In some embodiments, the length of a molecular moiety may be about 1, 2, 3, 4, 5, 6, 7, 8, 9, 10, 11, 12, 13, 14, 15 or more individual species or subunits. In some embodiments, the length of a molecular moiety can be useful in modulating the rate of a nucleic acid amplification reaction in which the molecular moiety participates.

In one embodiment, a molecular moiety of a primer or oligonucleotide described herein may comprise a nucleic acid. A molecular moiety of a primer or oligonucleotide described herein may reduce (or prevent) the ability of the primer or oligonucleotide to hybridize with another primer or oligonucleotide and or be extended in an amplification reaction.

In one embodiment, the molecular moiety of a 1st SW primer and a 2nd SW primer in a primer set may not be complementary to each other, such that the lack of sequence complementarity between the molecular moiety reduces (or prevents) the ability of the forward and reverse primers to hybridize to each other during an amplification reaction. The molecular moiety can be linked to a primer or oligonucleotide via one or more phosphodiester bonds that can be separated by ribose or deoxyribose and/or the molecular moiety can be terminated with a hydroxyl group.

In one embodiment, a molecular moiety of a primer or oligonucleotide described herein may be adapted such that its melting temperature is lower than the melting temperature of a portion of a nucleotide sequence of the primer or oligonucleotide. A lower melting temperature of a molecular moiety may reduce the likelihood (or prevent) binding of the molecular moiety to a target nucleic acid molecule at a primer or oligonucleotide annealing temperature that is higher than the melting temperature of the molecular moiety.

In one embodiment, the molecular moiety may comprise at least one, two, three, four, five, six, seven, eight, nine, ten or more nucleotides or nucleotide analogues. The molecular moiety may comprise one or more nucleotide analogues having an unnatural base. Non-limiting examples of nucleotide analogues having an unnatural base include inosine (including a base of hypoxanthine), uracil-containing nucleotides (in cases where a nucleic acid is DNA), iso-dC, iso-dG, diaminopurine, 2,4-difluoroloiuene, 4-methylbenzimidazole, size-expanded x.A, size-expanded xG, size-expanded xC, size-expanded xT, d5SICS and dNalv1. The molecular moiety may comprise one or more nucleotide analogues that have no base (e.g., abasic nucleotides, acyclo nucleotides). In some embodiments, a molecular moiety may comprise a terminator nucleotide that cannot be extended by a polymerase without removal (e.g., via an enzyme with proofreading activity, such as an exonuclease or endonuclease).

In one embodiment, a molecular moiety comprises at least an unit terminated with a hydroxyl group and comprises a nucleotide and/or a nucleotide analogue selected from a group comprising an inosine, a uracil-containing nucleotide, an iso-deoxycytosine (iso-dC), an iso-deoxyguanosine (iso-dG), a diaminopurine, 2,4-difluorotoluene, 4-methylbenzimidazole, a size-expanded adenine (xA), a size-expanded guanine (xG), a size-expanded cytosine (xC), a size-expanded thymine (xT), 2-((2R,4R,5R)-tetrahydro-4-hydroxy-5-(hydroxymethyl) furan-2-yl)-6-methylisoquinoline-1(2H)-thione (d5 SICS), 1,4-Anhydro-2-deoxy-1-C-(3-methoxy2-naphthalenyl)-(1R)-D-erythro-pentitol (dNaM), an abasic nucleotide, an acyclo nucleotides and/or combination thereof.

In one embodiment, the length of a molecular moiety that comprises nucleic acid may vary. For example, the length of a molecular moiety that comprises nucleic acid may be about 1, 2, 3, 4, 5, 6, 7, 8, 9, 10, 11, 12, 13, 14, 15 or more nucleotides or nucleotide analogues. In embodiments where a primer (including a primer of a primer set) or oligonucleotide (including an oligonucleotide of an oligonucleotide set) described here comprises a nucleotide sequence A that is complementary or substantially complementary to a target nucleic acid (including cases where nucleotide sequence A exhibits sequence complementarity to itself and/or a molecular moiety), a molecular moiety of the primer or oligonucleotide may comprise a nucleotide sequence B having 1-15, 1-10, 1-8, 1-6, or 1-4 consecutive nucleotides that are non-complementary with respect to 1-15, 1-10, 1-8, 1-6 or 1-4 corresponding nucleotides of a target nucleic acid molecule.

In one embodiment, a molecular moiety of a primer or oligonucleotide can be a substrate of an exonuclease, an endonuclease or both types of enzymes. Examples of exonucleases and endonucleases are described elsewhere herein. In such cases, an appropriate exonuclease and/or endonuclease can be used to remove the molecular moiety.

An embodiment relates to a removal or cleavage of a molecular moiety may be completed via the action of an enzyme (e.g., polymerase) with 3′-5′ exonuclease activity. Such an enzyme may “proofread” the molecular moiety such that individual species or subunits (e.g., non-complementary nucleotides) of the molecular moiety are removed one-by-one from the associated primer at its 3′ end in sequential fashion by the enzyme with 3′-5′ exonuclease activity. Any suitable enzyme with 3′-5′ exonuclease activity may be used to remove or cleave a molecular moiety from a 1st SW primer/a 2nd SW primer. Non-limiting examples of enzymes with 3′-5′ exonuclease activity include naturally occurring exonucleases, engineered exonucleases, Phusion polymerase, Pfu polymerase, DEEPVENT polymerase, exonuclease I, exonuclease III, exonuclease IV, exonuclease V, KOD polymerase, Q5 DNA polymerase, Advantage HD polymerase, PrimeST AR GXL DNA polymerase, Bst polymerase and Phi29 DNA polymerase. Typically, the 3′ moiety is more efficiently cleaved by a 3′-5′ exonuclease activity when it is present in a double-stranded nucleic acid molecule.

In one embodiment, removal of a molecular moiety from a 1st SW primer/a 2nd SW may be completed via the action of an enzyme (e.g., polymerase) with endonuclease activity. Such an enzyme may “proofread” the molecular moiety such that the entire molecular moiety is removed as a single species via, for example, the cleavage of a phosphodiester bond linking the molecular moiety to a primer. Any suitable enzyme with endonuclease activity may be used to remove or cleave a molecular moiety from a forward and/or reverse primer. Non-limiting examples of enzymes with endonuclease activity include naturally occurring endonucleases, engineered endonucleases, deoxyribonuclease I, Type I restriction endonucleases, Type II restriction endonucleases, Type III restriction endonucleases, thermal stable RNase HII, thermal stable RNase HI and thermal stable uracil DNA-glycosylase (UDG).

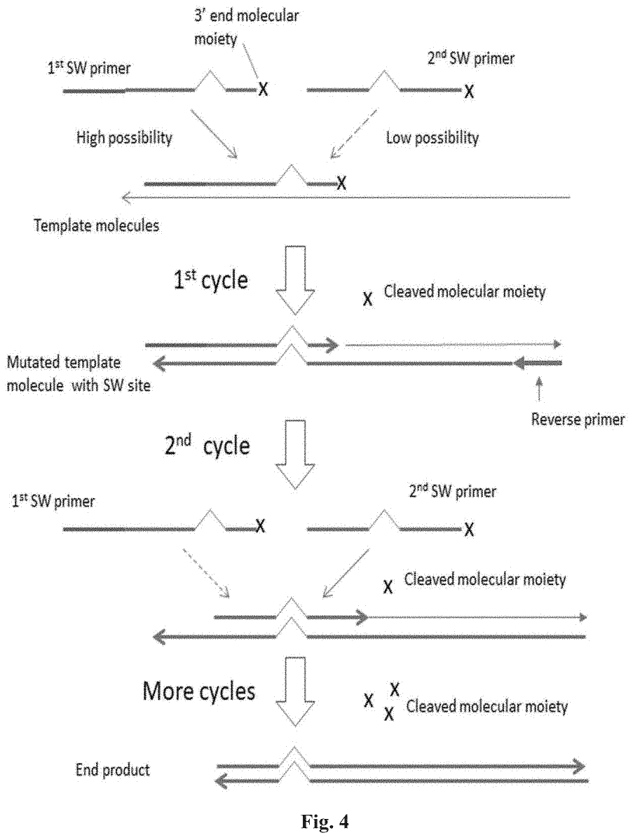

In one embodiment, the method of the single-tube nested PCR comprises (as illustrated in FIG. 4): annealing of the 1st SW primer with the target template molecules to form a stable hybrid; removal or cleavage of a molecular moiety of the primers via the action of an enzyme (e.g., polymerase) with 3′-5′ exonuclease activity; extending the 3′ end which is complementary to the target template molecule via the action of the polymerase to generate a mutated strand of the target template molecule. The mutated stand generated in the first cycle of single-tube nested PCR comprising the SW site of the 1st SW primer. In the next cycle of the single-tube nested PCR, the mutated strand of the target template molecule is copied using the reverse primer. Extension with reverse primer results in mutated complementary strand generation. The 2nd SW primer copies or amplifies the mutated complementary strand with high affinity.

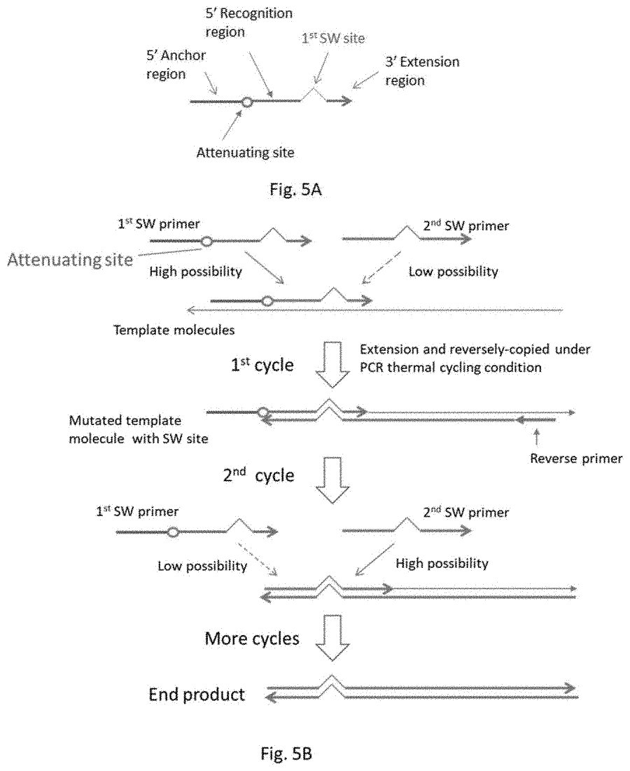

An embodiment relates to a 1st SW primer described herein may comprise an attenuating (or attenuation) site (as illustrated in FIG. 5A). The attenuating site is at 5′ region of the 1st SW primer, precisely downstream to the anchor sequence, preferably the attenuating site is located between the anchor sequence and the 5′ recognition sequence of the 1st SW primer. The attenuating site drastically influence DNA extension as an attenuating site blocks the extension by the polymerase. The attenuating site interrupts the extension of reverse primer by polymerase. Hence, a truncated mutant complementary strand is generated by the reverse primer extension which lacks the 5′anchor region of 1st SW primer. In some embodiments, the attenuating site reduces the possibility of annealing 1st SW primer to a truncated mutant complementary strand, as truncated mutant complementary strand lacks the 5′-anchor region. Hence the 1st SW primer could not form a stable hybrid with truncated mutant complementary strand, thereby increases the possibility of annealing of the 2nd SW primer to the truncated mutant complementary strand.

In one embodiment, the attenuating site increases specificity and sensitivity to identify/detect the target nucleotide sequence. The attenuating site decreases the 1st SW primer to mutant complementary strand binding efficiency, thereby preventing amplification of mutant complementary strand by 1st SW primer. Together with SW site and attenuating site, the ongoing unwanted amplification of the 1st SW primer is minimised and the amplification of the inner primer (2nd SW primer) can proceed which facilitates efficient amplification of the target sequence. In some embodiments, the length of the attenuating site of the 1st SW primer (including a primer of a primer set) or oligonucleotide (including an oligonucleotide of an oligonucleotide set) described herein may vary depending upon the particular primer or oligonucleotide. In some embodiments, the length of the attenuating site of a primer or oligonucleotide described herein may be from about 1 nucleotide to about 10 nucleotides long.

In one embodiment, the attenuating site comprise a modified nucleotide comprising a natural nucleotide, a non-natural nucleotide, an abasic site, a spacer, a fluorescent dye-modified nucleotides, an atypical nucleotide in DNA sequence comprised of deoxyuridine, a chemically synthesized nucleotide or combination thereof. An attenuating site can be an internal site or at the 3′ end. Therefore, an attenuating site of a primer or an oligonucleotide or a probe has the functional features of: (i) not cleavable by exonuclease, (ii) not directly extendable or not extended efficiently, (iii) if extended, not be copied due to non-complementary or strong polymerase binding. Example for strong polymerase binding to a nucleic acid unit is the archaebacterial polymerase binding to the nucleotide containing a uracil.

In one embodiment, the attenuating site may comprise at least one, two, three, four, five, six, seven, eight or more modified and non-natural nucleotides. In some embodiments, the attenuating site may comprise one or more modified nucleotides. Non-limiting examples of modified nucleotides include: N6-MedAMP, 6-Cl-PMP, 6-Cl-2APMP, O6-MedGMP, N2-MedGMP, 2-6-dAMP, dIMP and 8-oxo-dGMP or combination thereof. In some embodiments, the attenuating site may comprise one or more non-natural nucleotides. Non-limiting examples of non-natural nucleotides include: IndTP, 5-MeIMP, 5-Et-IMP, 5-EyIMP, 5-NIMP, 4-NIMP and 6-NIMP or combination thereof.

In one embodiment, the attenuating site may comprise at least one, two, three, four, five, six, seven, eight or more modified bases which are locked nucleic acids. Non-limiting examples of modified bases which are locked nucleic acids include: 2′-O-methoxy-ethyl bases, 2-MethoxyEthoxy A, 2-MethoxyEthoxy MeC, 2-MethoxyEthoxy G, 2-MethoxyEthoxy T, 2′-O-Methyl RNA Bases, Fluoro Bases, Fluoro C, Fluoro U, Fluoro A and Fluoro G, 8-aza-7-deazaguanosine, 2,6-Diaminopurine (2-Amino-dA), Dideoxy-C, Hydroxymethyl dC, Inverted dT, Iso-dG, Iso-dC, Inverted Dideoxy-T, Super T (5-hydroxybutynl-2′-deoxyuridine) and 5-Nitroindole or combination thereof. In one embodiment, the attenuating site may comprise at least one, two, three, four, five, six, seven, eight or more an atypical nucleotide in nucleic acid sequence which is substituted for purines and pyrimidines. Non-limiting examples of atypical nucleotide include: 5-Methyl dC, DeoxyUridine (dU), 5-Bromo-deoxyuridine (5-Bromo dU) and 2-Aminopurine or combination thereof.

In one embodiment, the method of the single-tube nested PCR comprises (as illustrated in FIG. 5B): annealing of the 1st SW primer comprising attenuating site with the template nucleic acid molecules to form a stable hybrid. Extension of the 3′ end which is complementary to the template occurs in a primer extension reaction via the action of the polymerase to generate a mutated complementary strand of the template. The mutated complementary stand generated in the first cycle of single-tube nested PCR comprising the SW site of the 1st SW primer. In the next cycle of the single-tube nested PCR, the mutated complementary strand of the template is copied using the reverse primer. Extension or amplification with a reverse primer results in mutated template strand generation. The truncated mutated template strand is generated which lacks 5′ anchor region of the 1st SW primer. The 2nd SW primer anneals to the truncated mutated template strand with high possibility and amplifies it.

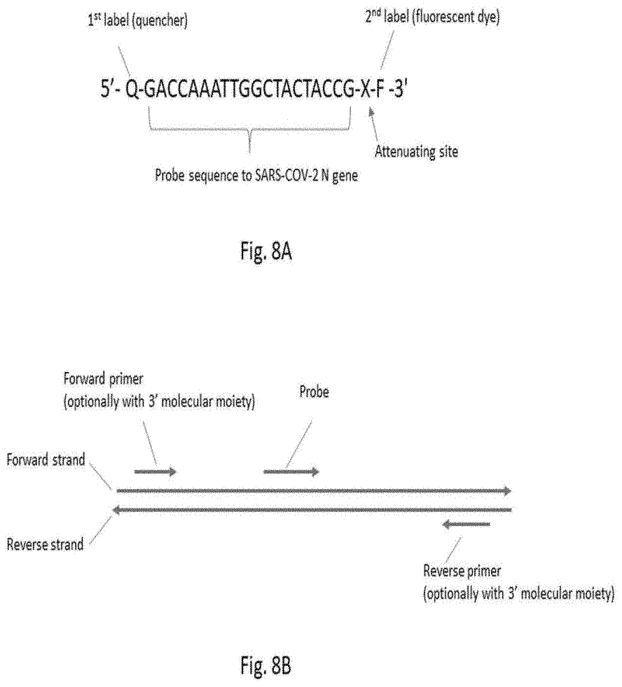

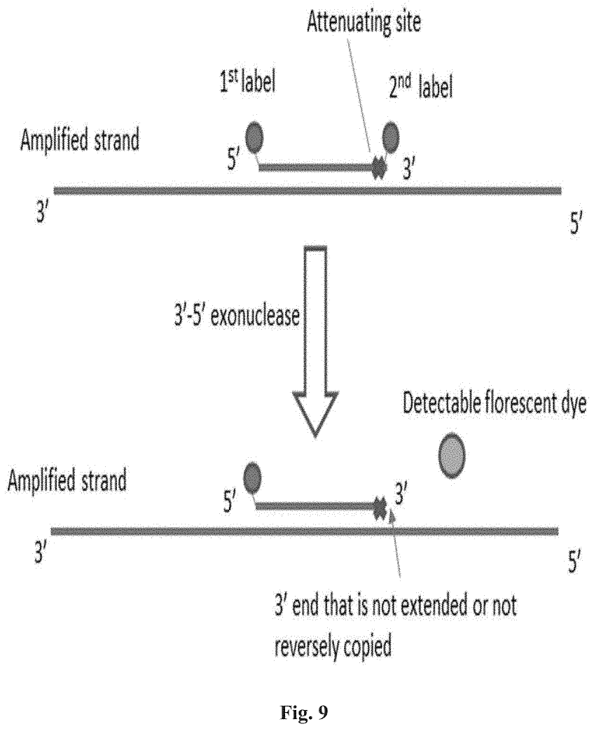

An embodiment relates to a method of the specific and/or quantitative detection of the nucleic acid sequence of interest comprises (as illustrated in FIG. 8): annealing an amplification forward primer to a strand of the target molecule comprising the nucleic acid sequence of interest; hybridizing the probe to a strand of the target molecule to form a probe:target duplex (as illustrated in FIG. 9); detecting a real-time increase in the emission of a signal after cleavage of a label off the probe using the 3′-5′ exonuclease activity of the polymerase (as illustrated in FIG. 8); extending the two primers using the target molecules as the templates; repeating the above steps to amplify and detect the target nucleic acid sequence.

In one embodiment, a probe comprising: (i) an attenuating site, (ii) a first label in a non 3′ site and (iii) a second label at the 3′ end. The second label at the 3′ end of the probe is cleavable effectively using the 3′-5′ exonuclease activity of the polymerase when the probe is hybridized to its target to form a double-stranded structure. A non 3′ site is a 5′ end position or an internal site that is 5′ upstream from the second label of the probe molecule. The second label is 3′ downstream from the first label, separated by 1-50 nucleotides. Thus, the second label at the 3′ end can be a label in the 3′ direction relative to the first label; it can be in the terminal position or in an internal site near the 3′ terminus comprising 1-3 units. The first label and the second label comprise a fluorescent dye-quencher pair or similar thereof. The attenuating site is located between the center of the probe and the second label. It is configured to prevent the probe molecules from being used as primers or templates to be copied to generate false positive signals in non-specific reactions. In one embodiment, the second label is placed immediately after the attenuating site (to the 3′ side of the attenuating site). Thus, cleavage of the second label at the 3′ end will expose the attenuating site. However, the attenuating site will not be extended efficiently or will not be copied if it is extended. A real-time increase in the emission of a signal can be detected after the cleavage of a label off the probe using the 3′-5′ exonuclease activity of the polymerase. The cleavage of a label, either the fluorescent dye or the quencher of the fluorescent dye: quencher pair, increases the molecular distance between the fluorescent dye moiety and the quencher moiety, resulting in less quenching or higher fluorescence emission. The cleavage of the second label at the 3′ end exposes the attenuating site that is not extended. As a result, the remaining portion of the probe will dissociate from its template under normal annealing conditions around respective Tm, allowing primer extension to continue to the end of the template strand. In one embodiment, the PCR primer concentrations are not significantly less but can be higher than the probe concentration in a PCR reaction, such as the primer:probe ratio is in the range of 1:2 to 10:1. When a lower probe concentration is used, the probe has a less chance to compete with the primer for the template molecules, allowing the PCR to proceed exponentially. In another embodiment, the exposed attenuating site may be extended, however, when the extended strand is being copied, the elongating strand will not pass the attenuating site, thus any product derived from the probe will not be amplified and thus the change to generate false positive signal is minimized. Taken together, the increase in fluorescence intensity is proportional to the amount of amplicon produced and the presence of the probe in a PCR reaction can be configured to ensure normal PCR reaction rate or efficiency. The single-tube quantitative nested PCR is more sensitive, generate stronger signals, take shorter time to complete and avoid non-specific PCR product.

An embodiment relates to a quantification and detection of target nucleic acid sequences present in template nucleic acid molecules by quantitative PCR. More specifically, the embodiments provide, in part, methods for single-tube nested qPCR of a target nucleic acid sequence, the polymerase can cleave the probe to free the label or dye out from the probe or primer to generate the signal.

In one embodiment, a reaction mixture for single-tube nested qPCR comprises a forward primers, a probe, a reverse primer, and a template nucleic acid molecule including a target region, polymerase with 3′-5′ exonuclease activity, a set of dNTP's and a buffer system. The forward primers can be a 1st SW primer and a 2nd SW primer. The forward primers, the probe and the reverse primer are useful for amplification of the template and the signal generation. The reaction mixture can additionally include an outer primer, where the outer primer configured to amplify the templates nucleic acid molecules for the 1st SW primer. The outer primer is used to increase the detection sensitivity by provide more template molecules for forward primers (1st SW primer and 2nd SW primer)

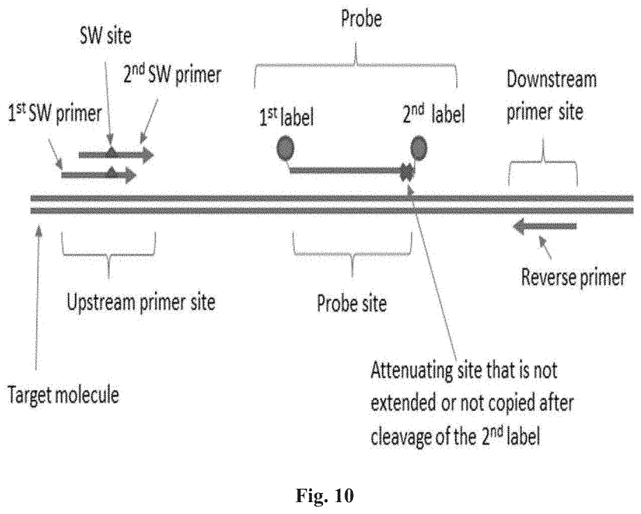

In one embodiment, the method of the single-tube quantitative nested PCR (as illustrated in FIG. 10) comprises: annealing of the 1st SW primer with the target molecules to form a stable hybrid. Extension of the 3′ end which is complementary to the template occurs in a primer extension reaction via the action of the polymerase to generate a mutated strand of the target template molecule. The mutated strand generated in the first cycle of single-tube quantitative nested PCR comprises the SW site of the 1st SW primer. In the next cycle of the single-tube quantitative nested PCR, the mutated strand of the template is copied using the reverse primer. Extension or amplification with reverse primer results in mutated complementary strand generation. The 2nd SW primer copies or amplifies the mutated complementary strand. The probe with an attenuating site can hybridize to a strand of the target molecule to form a probe: target duplex. The attenuating site is located between the center of the probe and the second label. It is configured to prevent the probe molecules from being used as primers or templates to be copied to generate false positive signals in non-specific reactions. A real-time increase in the emission of a signal can be detected after the cleavage of a label off the probe using the 3′-5′ exonuclease activity of the polymerase. The cleavage of a label, either the fluorescent dye or the quencher of the fluorescent dye: quencher pair, increases the molecular distance between the fluorescent dye moiety and the quencher moiety, resulting in less quenching or higher fluorescence emission. The cleavage of the second label at the 3′ end exposes the attenuating site that is not extended. As a result, the remaining portion of the probe will dissociate from its template, allowing primer extension to continue to the end of the template strand.

In one embodiment, the PCR primer concentrations are not significantly less but can be higher than the probe concentration in a PCR reaction, such as the primer:probe ratio is in the range of 1:2 to 10:1. When a lower probe concentration is used, the probe has a less chance to compete with the primer for the template allowing the PCR to proceed exponentially.

In another embodiment, the exposed attenuating site may be extended, however, when the extended strand is being copied, the elongating strand will not pass the attenuating site, thus any product derived from the probe will not be amplified and thus the change to generate false positive signal is minimized. Taken together, the increase in fluorescence intensity is proportional to the amount of amplicon produced and the presence of the probe in a PCR reaction can be configured to ensure normal PCR reaction rate or efficiency. The single-tube quantitative nested PCR is more sensitive, generate stronger signals, take shorter time to complete and avoid non-specific PCR product.

In one embodiment, quantitative PCR or qPCR monitors the fluorescence emitted during the reaction as an indicator of amplicon production during PCR, as opposed to endpoint detection. The real-time progress of the reaction can be viewed in some systems.

An embodiment relates to a method of the specific and/or quantitative detection of the nucleic acid sequence of interest comprises: annealing of an amplification primer to a strand of the target molecule comprising the nucleic acid sequence of interest; amplifying the two strands of the target molecule between the first and second amplification primer sites in the presence of the polymerase; hybridizing the probe to a strand of the target molecule to form a probe:target duplex; detecting a real-time increase in the emission of a signal by cleavage of a label off the probe using the 3′-5′ exonuclease activity of the polymerase (as illustrated in FIG. 8).

In one embodiment, quantitative PCR uses the detection of a fluorescent reporter. Typically, the fluorescent reporter's signal increases in direct proportion to the amount of PCR product in a reaction. By recording the amount of fluorescence emission at each cycle (conventional thermal cycling) or during the reaction (convective thermal cycling), it is possible to monitor the PCR reaction during exponential phase where the first significant increase in fluorescent signal (determined by Ct or inflection point) correlates to the initial amount of target template. The higher the starting copy number of the nucleic acid target, the sooner (fewer Ct number or shorter time for inflection point) a significant increase in fluorescence is observed.

In one embodiment, quantitative PCR uses multiple probe-based assays, in which each assay has a specific probe labeled with a unique fluorescent dye, resulting in a different observed color for each assay. qPCR instruments can discriminate between the fluorescence generated from different dyes. Different probes can be labeled with different dyes that each has a unique emission spectrum. Spectral signals are collected with discrete optics, passed through a series of filter sets, and collected by an array of detectors. Spectral overlap between dyes may be corrected by using pure dye spectra to deconvolute the experimental data by matrix algebra.

In one embodiment, the fluorescently-labeled probes (such as probes disclosed herein) rely upon fluorescence resonance energy transfer (FRET), or in a change in the fluorescence emission wavelength of a sample, as a method to detect hybridization of a DNA probe to the amplified target nucleic acid in real-time. For example, FRET that occurs between fluorogenic labels on different probes (for example, using HybProbes) or between a donor fluorophore and an acceptor or quencher fluorophore on the same probe (for example, using a molecular beacon or a TaqMan® probe) can identify a probe that specifically hybridizes to the DNA sequence of interest and in this way, using a M. pneumoniae CARDS toxin probe, a C. pneumoniae ArgR probe, and/or a Legionella spp.

In one embodiment, FRET (Fluorescence Resonance Energy Transfer) is a spectroscopic process by which energy is passed between an initially excited donor to an acceptor molecule separated by 10-100 Å. The donor molecules typically emit at shorter wavelengths that overlap with the absorption of the acceptor molecule. The efficiency of energy transfer is proportional to the inverse sixth power of the distance (R) between the donor and acceptor (1/R6) fluorophores and occurs without emission of a photon. In applications using FRET, the donor and acceptor dyes are different, in which case FRET can be detected either by the appearance of sensitized fluorescence of the acceptor or by quenching of donor fluorescence. For example, if the donor's fluorescence is quenched it indicates the donor and acceptor molecules are within the Forster radius (the distance where FRET has 50% efficiency, about 20-60 Å), whereas if the donor fluoresces at its characteristic wavelength, it denotes that the distance between the donor and acceptor molecules has increased beyond the Forster radius, such as when a TAQMAN® probe is degraded by Taq DNA polymerase following hybridization of the probe to a target nucleic acid sequence or when a hairpin probe is hybridized to a target nucleic acid sequence. In another example, energy is transferred via FRET between two different fluorophores such that the acceptor molecule can emit light at its characteristic wavelength, which is always longer than the emission wavelength of the donor molecule.

In one embodiment, examples of oligonucleotides using FRET that can be used to detect amplicons include linear oligoprobes, such as HybProbes, 5′ nuclease oligoprobes, such as TAQMAN® probes, hairpin oligoprobes, such as molecular beacons, scorpion primers and UniPrimers, minor groove binding probes, and self-fluorescing amplicons, such as sunrise primers or LUX primers.

In one embodiment, the fluorescently-labeled DNA probes used to identify amplification products have spectrally distinct emission wavelengths, thus allowing them to be distinguished within the same reaction tube. e.g., TaqMan probes, TaqMan Tamara probes, TaqMan MGB probes, Lion probes, molecular beacons).

In one embodiment, an additional method of detection that can be used to detect a nucleic acid molecule described herein is a melting curve analysis. In particular, a melting curve analysis may be useful in detecting primer dimer molecular complexes or by-products and/or single nucleotide polymorphisms, as described elsewhere herein. In a melting curve analysis, a mixture (e.g., an amplification reaction mixture) comprising double-stranded nucleic acid molecules can be heated and dissociation (e.g., denaturing) of the double-stranded nucleic acid molecules in the mixture can be measured against temperature. The temperature dependent dissociation of strands of a double-stranded nucleic molecule can be measured using a detectable species (e.g., a fluorophore such as, for example SYBR green or EvaGreen, nucleic acid probes labeled with a detectable species) that can intercalate or bind double stranded nucleic acid molecules. For example, in the case of an intercalator (e.g., SYBR green) that fluoresces when bound to a double-stranded nucleic acid molecule, the dissociation of double-stranded nucleic acid molecules during heating can be determined by a reduction in fluorescence that results. A reduction in fluorescence can result due to the release of an intercalating dye from a dissociated double-stranded nucleic acid molecule. The free dye may not fluoresce (or may not fluoresce at the same wavelength as the bound species) and thus, a reduction in fluorescence may be used to indicate a dissociation of double-stranded nucleic acid molecules. The first derivative or negative first derivative of dissociation (e.g., negative first derivative of fluorescence) as a function of temperature may be plotted to determine a temperature of dissociation (e.g., temperature at which 50% dissociation occurs) via peaks in the plot. A nucleic acid molecule may be identified via the obtained dissociation profile and/or temperature of dissociation.

In one embodiment, a melting curve analysis of the amplified target nucleic acid can be performed subsequent to the amplification process. The Tm of a nucleic acid sequence depends on the length of the sequence and its G/C content. Thus, the identification of the Tm for a nucleic acid sequence can be used to identify the amplified nucleic acid, for example by using double-stranded DNA binding dye chemistry, which quantitates the amplicon production by the use of a non-sequence specific fluorescent intercalating agent (such as SYBR® Green or ethidium bromide). SYBR® Green is a fluorogenic minor groove binding dye that exhibits little fluorescence when in solution but emits a strong fluorescent signal upon binding to double-stranded DNA. Typically, SYBR® Green is used in single plex reactions, however when coupled with melting point analysis, it can be used for multiplex reactions.

In one embodiment, PCR systems generally rely upon the detection and quantitation of fluorescent dyes or reporters, the signal of which increase in direct proportion to the amount of PCR product in a reaction. For example, in the simplest and most economical format, that reporter can be the double-strand DNA-specific dye SYBR® Green (Molecular Probes). SYBR Green is a dye that binds the minor groove of double stranded DNA. When SYBR Green dye binds to a double stranded DNA, the fluorescence intensity increases. As more double stranded amplicons are produced, SYBR Green dye signal will increase.

In one embodiment, non-limiting examples of DNA-specific dyes include SYBR green, SYBR blue, DAPI, propidium iodine, Hoeste, SYBR gold, EvaGreen, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), SYBR Green, Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In one embodiment, an amplified nucleic acid molecule described herein (e.g., including an amplified product of a target nucleic acid molecule, an amplified product of a nucleic acid sample, and an amplified double-stranded target nucleic acid molecule described elsewhere herein) may be detected at varied specificity. For example, specificity may depend on the particular primers used for amplification, nucleic acid molecule to be amplified, and/or other species in an amplification reaction mixture. An example measure of amplification specificity is the cycle threshold (Ct) for an amplification product during an amplification reaction, as described elsewhere herein. In some embodiments, Ct value can be anywhere between the total number of cycles of a given amplification reaction and any number above background level. In some embodiments, Ct value can be inversely proportional to the initial amount of a nucleic acid molecule to be amplified. For example, the cycle threshold for an amplified nucleic acid molecule obtained using a method for nucleic acid amplification described herein may be detected at a Ct value of less than 80, 75, 70, 65, 60, 55, 50, 45, 40, 35, 30, 25, 20, 15, 10, 5 or less.

In another embodiment, any type of thermal cycler apparatus can be used for the amplification or the determination of hybridization. Examples of suitable apparatuses include PTC-100® Peltier Thermal Cycler (MJ Research, Inc.; San Francisco, Calif.), a RoboCycler® 40 Temperature Cycler (Agilent/Stratagene; Santa Clara, Calif.), or GeneAmp® PCR System 9700 (Applied Biosystems; Foster City, Calif.). For qPCR, any type of quantitative thermocycler apparatus can be used. For example, iCycler iQTM or CFX96™ real-time detection systems (Bio-Rad, Hercules, Calif.), LightCycler® systems (Roche, Mannheim, Germany), a 7700 Sequence Detector (Perkin Elmer/Applied Biosystems; Foster City, Calif.), ABI™ systems such as the 7000, 7300, 7500, 7700, or 7900 systems (Applied Biosystems; Foster City, Calif.), or an MX4000TM, MX3000TM or MX3005TM qPCR system (Agilent/Stratagene; Santa Clara, Calif.), DNA Engine Opticon® Continuous Fluorescence Detection System (Bio-Rad, Hercules, Calif.), Rotor-Gene® Q real-time cycler (Qiagen, Valencia, Calif.), or SmartCycler® system (Cepheid, Sunnyvale, Calif.) can be used to amplify nucleic acid sequences in real-time. In some embodiments qPCR is performed using a TaqMan® array format, for example, a microfluidic card in which each well is pre-loaded with primers and probes for a particular target. The reaction is initiated by adding a sample including nucleic acids and assay reagents (such as a PCR master mix) and running the reactions in a quantitative thermocycler apparatus.

In some embodiments, a convective thermal device is used for PCR. In such a device temperature control is resulted from liquid convection due to density gradients. In a convective thermal device, the lower portion of a test tube or vessel containing a reaction mixture is set to a temperature higher (lower liquid density) than that of the upper portion of the same test tube or vessel (higher liquid density). An optical system can be configured to detect fluorescent signal from the reaction tubes or vessels in a convective thermal device.

In some embodiments, the probe is detectably labeled, either with an isotopic or non-isotopic label; in alternative embodiments, the target nucleic acid is labeled. Non-isotopic labels can, for instance, comprise a fluorescent or luminescent molecule, or an enzyme, co-factor, enzyme substrate, or hapten. The probe is incubated with a single-stranded or double-stranded preparation of RNA, DNA, or a mixture of both, and hybridization is determined. In some examples, the hybridization results in a detectable change in signal such as in increase or decrease in signal, for example from the labeled probe. Thus, detecting hybridization comprises detecting a change in signal from the labeled probe during or after hybridization relative to signal from the label before hybridization.

An embodiment relates to a target nucleotide sequence might be present in a biological or a non-biological sample. Examples of non-biological samples includes, for example, the nucleic acid products of synthetically produced nucleic acid populations. Reference to a “biological sample” should be understood as a reference to any sample of biological material derived from an animal, plant or microorganism (including cultures of microorganisms) such as, but not limited to, cellular material, blood, mucus, faeces, urine, tissue biopsy specimens, fluid which has been introduced into the body of an animal and subsequently removed (such as, for example, the saline solution extracted from the lung following lung lavage or the solution retrieved from an enema wash), plant material or plant propagation material such as seeds or flowers or a microorganism colony. The biological sample which is tested according to the method aforementioned embodiments may be tested directly or may require some form of treatment prior to testing. For example, a biopsy sample may require homogenisation prior to testing. Further, to the extent that the biological sample is not in liquid form, it may require the addition of a reagent, such as a buffer, to mobilise the sample.

An embodiment relates to a target nucleotide sequence might be the RNA or DNA sequence of a pathogen. The target molecule could be single strand or double strand. RNA pathogens include, but are not limited to RNA viruses, and DNA pathogens include, but are not limited to DNA viruses. Examples of RNA viruses include the Togavirus family of RNA viruses, which includes the genus alphavirus, which in turn, includes many important viral species such as Sindbis virus, Semliki Forest virus, and pathogenic members such as the Venezuelan, Eastern and Western equine encephalitis virus. Another pathogenic Togavirus is the rubella virus, a virus closely related to the alphaviruses and the causative agent for German measles. Coronaviruses (which includes SARS-CoV-2) and astroviruses (associated with pediatric diarrhea) are also pathogenic RNA viruses. The Picornaviruses are also RNA viruses which include the Poliovirus, Coxsackievirus, Echovirus, Enterovirus and Rhinovirus. DNA viruses include Paroviruses, Papovaviruses which include the Papilloma viruses which can infect rabbits and the Polyomaviruses which infect primates, Adenoviruses, Herpes viruses, and hepadna viruses. Others are known in the art.

In one embodiment, DNA pathogens include microbes such as bacteria and yeast. Exemplary microbes include the Bacillus, Chlamydia, and Streptococcus species. The genome sequences of microbes are publicly available at www.ncbi.nlm.nih.gov/genomes/MICROBES/complete.html. Retroviruses may also be detected by the method of the invention. A retrovirus is an RNA virus that has a DNA intermediate step during replication. Retroviruses include the human immunodeficiency virus (HIV). Others are known in the art and sequences of various retrovirus genomes can be found at www.ncbi.nlm.nih.gov/retroviruses/.

In one embodiment, a nucleic acid amplification method of present disclosure allows for direct amplification of a sample, without the step of nucleic acid isolation.

An embodiment relates to quantification and detection of target nucleic acid sequences present in template nucleic acid molecules by single-tube-nested qPCR. More specifically, the embodiments provide, in part, methods for single-tube nested qPCR of a target nucleic acid sequence, wherein at least one primer has fluorescent probe at its 3′-end and a quencher at its 5′-end. The probe primer can be used together with polymerase with 3′-5′ exonuclease activity. The polymerase can cleave the probe primer to free the dye out from the primer to generate the signal.

In one embodiment, a reaction mixture for single-tube nested qPCR comprises a forward primer, a forward probe primer, a reverse primer, and a template nucleic acid molecule including a target region, polymerase with 3′-5′ exonuclease activity, a set of dNTP's and a buffer system. The forward primer is also known as a 1st SW primer. The forward probe primer also known as a 2nd SW probe primer. The forward primer, the forward probe primer and the reverse primer are useful for amplification of the template and the signal generation. The reaction mixture can additionally include an outer primer, where an outer primer is configured to amplify the templates nucleic acid molecules for the 1st SW primer and the 2nd SW probe primer. The outer primer is used to increase the detection sensitivity by provide more template molecules for forward primers.

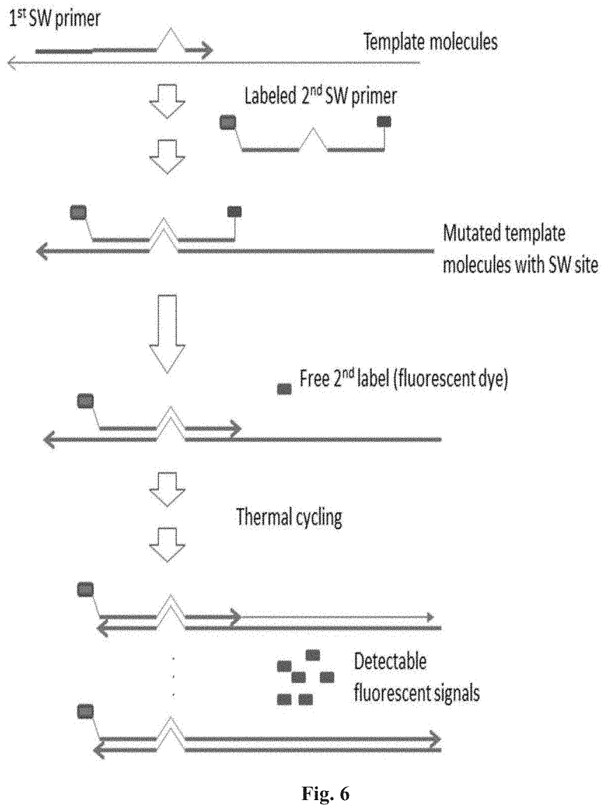

In one embodiment, a 2nd SW probe primer comprises (i) a 5′ recognition region, (ii) a long 3′ recognition region, (iii) a selective wobbling site wherein the second selective wobbling site is close to a central region, (iv) a first label in a non 3′ site (v) a second label at the 3′ end (as illustrated in FIG. 6A).

In one embodiment, the method of the single-tube nested qPCR (as illustrated in FIG. 6B) comprises: annealing of the 1st SW primer with the template nucleic acid molecules to form a stable hybrid. Extension of the 3′ end which is complementary to the template occurs in a primer extension reaction via the action of the polymerase to generate a mutated complementary strand of the template. The mutated complementary stand generated in the first cycle of single-tube quantitative nested PCR comprises the selective wobbling site of the 1st SW primer. In the next cycle of the single-tube quantitative nested PCR, the mutated complementary strand of the template is copied using the reverse primer. The 2nd SW probe primer amplifies the mutated complementary strand of the template amplified by the reverse primer. The polymerase can cleave the probe primer to free the dye out from the primer to generate the signal.

An embodiment relates to a nucleic acid amplification may occur in a reaction mixture in which the nucleic acid molecule to be amplified is provided along with any additional reagents (e.g. one or more of forward primers, reverse primers, polymerases, exonuclease, endonuclease, dNTPs, co-factors, suitable buffers, etc.) necessary for amplification of the nucleic acid molecule. Other reagents (e.g., a detectable species such as a probe or dye) may also be included in a reaction mixture that may be useful for the detection of an amplification product. The reaction mixture may then be subjected to conditions (e.g., appropriate temperatures, addition/removal of heat, buffer concentrations, etc.) suitable for amplifying the nucleic acid molecule. For example, a single or double-stranded target nucleic acid molecule may be provided in a reaction mixture that also comprises any additional reagents (e.g., one or more of a forward primers and reverse primers described elsewhere herein, a polymerase, an exonuclease, an endonuclease, random primers, dNTPs, co-factors, buffers, other enzymes (e.g., a reverse transcriptase to generate cDNA from RNA, a ligase, etc.) necessary for amplification of the single or double-stranded target nucleic acid molecule.

In one embodiment, salts and buffers include those familiar to those skilled in the art, including those comprising MgCl2, MgSO4, Tris-HCl, NaCl, KCl, and K2SO4, and other ingredients necessary for a PCR reaction. Typically, 1.5-3.0 mM of magnesium ion is optimal for DNA polymerase, however, the optimal magnesium ion concentration may depend on template, buffer, DNA and dNTPs as each has the potential to chelate magnesium ions. If the concentration of magnesium ion [Mg2+] is too low, a PCR product may not form. If the concentration of magnesium ion [Mg2+] is too high, undesired PCR products may be seen. In some embodiments the magnesium ion concentration may be optimized by supplementing magnesium ion concentration in 0.1 mM or 0.5 mM increments up to about 5 mM.

In one embodiment, buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In one embodiment, the temperature of the reaction mixture may be cycled repeatedly through a denaturation temperature (e.g., to denature, separate or melt double-stranded nucleic acid molecules into component nucleic acid strands), an annealing temperature (e.g., to anneal or hybridize a primer to each of the component nucleic acid strands) and an extension temperature (e.g., to extend or add nucleotides to the annealed primers in a primer extension reaction via the action of a polymerase) in order to amplify the single-stranded or double-stranded target nucleic acid molecule. For PCR performed in a convective thermal device, the denaturation temperature, annealing temperature and extension temperature are fixed for different regions of a reaction vessel. The aqueous reaction mixture, instead, goes through the temperature cycling by convection.

In one embodiment, the cycling of the temperatures of a reaction mixture may be achieved, for example, with the aid of any suitable thermocycler instrument or other type of device capable of cyclical heating. Such an instrument may include or may be coupled to a device suitable of detecting amplification products in a reaction mixture, as described elsewhere herein. In some embodiments, such a device may be capable of optically detecting an optically-responsive species in a reaction mixture, where such optical detection can be used for quantification of amplification products, measurement of Ct values, and/or melting point detection. In some embodiments, detection of amplified products can be performed in real-time (e.g., as the amplification reaction proceeds). In some embodiments, denaturation of a double-stranded nucleic acid molecule may be achieved via a denaturing agent, such as, for example an alkaline agent (e.g. sodium hydroxide (NaOH)).

In one embodiment, amplification and detection, using probe as described elsewhere herein, of a nucleic acid may be achieved isothermally such as, for example, without a change in temperature setting of the device. Isothermal amplification methods including, for example, the amplification methods of LAMP (Loop-mediated isothermal amplification), RPA (Recombinase Polymerase Amplification). In some embodiments, a method for nucleic acid amplification described herein may be completed without cycling the temperature of an amplification reaction mixture. For example, multiple amplification cycles may be performed without cycling the temperature of a reaction mixture.

In one embodiment, a probe comprising: (i) an attenuating site, (ii) a first label in a non 3′ site and (iii) a second label at the 3′ end. A non 3′ site is a 5′ end position or an internal site that is 5′ upstream from the second label of the probe molecule. The second label is 3′ downstream from the first label, separated by 1-50 nucleotides. Thus, the second label at the 3′ end can be a label in the 3′ direction relative to the first label; it can be in the terminal position or in an internal site near the 3′ terminus comprising 1-3 units. The second label at the 3′ end label of the probe is cleavable effectively using a 3′-5′ exonuclease when the probe is hybridized to its target to form a double-stranded structure. The first label and the second label comprise a fluorescent dye-quencher pair or similar thereof. In one embodiment, the second label is placed immediately 3′ to the attenuating site. The attenuating site is located between the center of the probe and the second label. It is configured to prevent the probe molecules from being used as primers or templates to be copied to generate false positive signals in non-specific reactions. A real-time increase in the emission of a signal can be detected after the cleavage of a label off the probe using the 3′-5′ exonuclease. The cleavage of a label, either the fluorescent dye or the quencher of the fluorescent dye: quencher pair, increases the molecular distance between the fluorescent dye moiety and the quencher moiety, resulting in less quenching or higher fluorescence emission.

In one embodiment, a reaction mixture may be heated to one or more reaction temperatures via the aid of a thermal gradient. The thermal gradient may, for example, be generated by one or more isothermal heating sources or one or more fixed heating sources, collectively called a convective thermal device herein. For example, a reaction mixture may be heated in a convection-based thermal gradient instrument such as, for example, via Rayleigh-Bernard convection. Such an instrument may include or may be coupled to a device suitable of detecting amplification products in a reaction mixture, as described elsewhere herein. In some embodiments, such a device may be capable of optically detecting an optically responsive species in a reaction mixture, where such optical detection can be used for quantification of amplification products, and/or melting point detection. In some embodiments, detection and displaying the signal of amplified products via convection-based strategies and/or instruments can be performed in real-time (e.g., as the amplification reaction proceeds), including detection via detection of melting points.

In one embodiment, a convection-based strategy and system include the iiPCR method used in a POCKIT system. Such a system can include a single heating source at the bottom of one or more vessels (e.g., capillary tubes) that drives an amplification reaction via Rayleigh-Bernard convection. When Rayleigh-Bernard convection is used to drive an amplification reaction, the temperature changes of different “parts” of the reaction mixture are generally not synchronized. In such cases, different parts of a reaction mixture in a reaction vessel can have different temperatures. Such temperature differences can be, between the highest and the lowest point, as large as 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C. or up to 60° C. or more. Moreover, a region of the reaction mixture can be moved to different regions of the reaction vessel due to temperature-related density differences among different regions. An additional feature of Rayleigh-Bernard convection-based amplification is that each given part of a reaction mixture can undergo continuous temperature changes along a temperature gradient that is generated by one or more isothermal heating sources. Such temperature changes can permit amplification of a nucleic acid molecule rapidly using an isothermal heating device.

An embodiment relates to a nucleic acid amplification reaction can include the use and action of a polymerase. During a primer extension reaction, a polymerase can generally add, in template-directed fashion, nucleotides to the 3′ end of a primer annealed to a single-stranded nucleic acid molecule. Any suitable polymerase may be used for a primer extension reaction, including commercially available polymerases. Non-limiting examples of polymerases include Taq DNA polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poe polymerase, Pab polymerase, Mth polymerase, Pho polymerase, Phusion polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, Q5 DNA polymerase, Advantage HD Polymerase, PrimeSTAR GXL DNA polymerase, polymerases with 3′-5′ exonuclease activity, and variants, modified or recombinant products and derivatives thereof.

In one embodiment, a suitable denaturation temperature may be, for example, about 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 93° C., 94° C., 95° C., 96° C., 97° C., 98° C., 99° C., 100° C., 101° C., 102° C., 103° C., 104° C., 105° C. or higher. In some embodiments, a suitable denaturation time for a single amplification cycle may be, for example, about 0.1 seconds (“s), 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 33 s, 34 s, 35 s, 36 s, 37 s, 38 s, 39 s, 40 s, 41 s, 42 s, 43 s, 44 s, 45 s, 46 s, 47 s, 48 s, 49 s, 50 s, 51 s, 52 s, 53 s, 54 s, 55 s, 56 s, 57 s, 58 s, 59 s, 1 minute, 2 minutes, 3 minutes, 4 minutes, 5 minutes or longer.

In one embodiment, a suitable annealing temperature may be, for example, about 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., or higher. In some embodiments, a suitable annealing time for a single amplification cycle may be, for example, about 0.1 s, 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 33 s, 34 s, 35 s, 36 s, 37 s, 38 s, 39 s, 40 s, 41 s, 42 s, 43 s, 44 s, 45 s, 46 s, 47 s, 48 s, 49 s, 50 s, 51 s, 52 s, 53 s, 54 s, 55 s, 56 s, 57 s, 58 s, 59 s, 1 minute, 2 minutes, 3 minutes, 4 minutes, 5 minutes or longer.

In one embodiment, a suitable extension temperature may be, for example, about 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., or higher. In some embodiments, a suitable extension temperature may be the same temperature as a suitable annealing temperature. In some embodiments, a suitable extension time for a single amplification cycle may be, for example, about 0.1 s, 0.5 s, 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s, 20 s, 21 s, 22 s, 23 s, 24 s, 25 s, 26 s, 27 s, 28 s, 29 s, 30 s, 31 s, 32 s, 33 s, 34 s, 35 s, 36 s, 37 s, 38 s, 39 s, 40 s, 41 s, 42 s, 43 s, 44 s, 45 s, 46 s, 47 s, 48 s, 49 s, 50 s, 51 s, 52 s, 53 s, 54 s, 55 s, 56 s, 57 s, 58 s, 59 s, 1 minute, 2 minutes, 3 minutes, 4 minutes, 5 minutes or longer.

An embodiment relates to a nucleic acid amplification reaction may be used to amplify a nucleic acid molecule. One example of a nucleic acid amplification reaction is a polymerase chain reaction (PCR) that relies on repeated cycles of primer annealing, primer extension and denaturing of amplified nucleic acid molecules as described above. Additional non-limiting examples of types of nucleic acid amplification reactions include reverse transcription, in vitro transcription, ligase chain reaction, nested amplification, multiplex amplification, helicase-dependent amplification, asymmetric amplification, rolling circle amplification, multiple displacement amplification (MDA); and variants of PCR that include qPCR, hot start PCR, inverse PCR, methylation-specific PCR, allele-specific PCR, assembly PCR, asymmetric PCR, miniprimer PCR, multiplex PCR, nested PCR, overlap-extension PCR, digital PCR, emulsion PCR, dial-out PCR, helicase-dependent PCR, nested PCR, thermal asymmetric interlaced PCR, single-tube PCR, quantitative PCR, multiple PCR, direct PCR and touchdown PCR.

An embodiment relates to a method for nucleic acid amplification described herein may include a reverse transcription polymerase chain reaction (RT-PCR). An RT-PCR nucleic acid amplification reaction may include the use of a reverse transcriptase and a reverse transcription primer or random primers that can generate complementary DNA (cDNA) from an RNA template. The cDNA can then be amplified with appropriate forward and reverse primers and the action of polymerase in a PCR nucleic acid amplification reaction. Thus, a reaction mixture in which an RT-PCR nucleic acid amplification reaction takes place may include a reverse transcriptase. Any suitable reverse transcriptase may be used for an RT-PCR nucleic acid amplification reaction with non-limiting examples of reverse transcriptases that include HIV-1 reverse transcriptase, M-MLV reverse transcriptase, AMV reverse transcriptase, telomerase reverse transcriptase, and variants, modified products and derivatives thereof. In cases where forward and/or reverse primers include molecular moieties, an RT-PCR reaction mixture may also include an enzyme capable of cleaving the molecular moiety, such as, for example, an enzyme having 3′-5′ exonuclease activity, as described elsewhere herein. The presence of molecular moieties in a 1st SW primer and a 2nd SW primer/a 2nd SW primer of an RT-PCR amplification reaction can increase the sensitivity of an RT-PCR amplification reaction such as, for example, by inhibiting mis-priming by a reverse transcriptase.

In one embodiment, an RT-PCR nucleic acid amplification reaction can be performed in a single reaction mixture (e.g., such as a reaction mixture in a single vessel), where all reagents (e.g., RNA template, dNTPs, polymerase, reverse transcriptase, enzyme with 3′-5′ exonuclease activity, reverse transcription primer, a probe, a forward primer and a reverse primer etc.) necessary to generate cDNA from an RNA template and further amplify the generated cDNA are provided in the reaction mixture. The reaction mixture can be subject to appropriate conditions (e.g., temperatures, etc.) to complete the various phases (e.g., reverse transcription of an RNA template to generate cDNA, amplification of the cDNA, etc.) of the RT-PCR amplification reaction. The entire RT-PCR amplification reaction can proceed without removal or addition of further reagents or contents to the reaction mixture.

An embodiment relates to a method may comprise detecting one or more nucleic acid molecules described herein, such as, for example amplified double-stranded target nucleic acid molecules, amplified products of a nucleic acid sample, amplified products of a target nucleic acid molecule, double-stranded nucleic acid molecules, single-stranded nucleic acid molecules, target nucleic acid molecules, forward primers, reverse primers, and/or primer dimer molecular complexes or by-products. The method described herein may comprise detecting at least a subset of amplified double-stranded target nucleic acid molecules, amplified products of a nucleic acid sample, or amplified products of a target nucleic acid molecule. Detection of any type of nucleic acid molecule described herein may be achieved via any suitable detection method or modality. The particular type of detection method or modality used may depend, for example, on the particular species being detected, other species present during detection, whether or not a detectable species is present, the particular type of detectable species to-be-used and/or the particular application.

In one embodiment, detection methods include optical detection, spectroscopic detection, electrostatic detection and electrochemical detection. Accordingly, a nucleic acid molecule described herein may be detected by detecting signals (e.g., signals indicative of an optical property, a spectroscopic property, an electrostatic property or an electrochemical property of the nucleic acid molecule or an associated detectable species) that are indicative of the presence or absence of the nucleic acid molecule. Optical detection methods include, but are not limited to, fluorescence emission detection, visual inspection (e.g., detection via the eye, observing an optical property or optical event without the aid of an optical detector), fluorimetry, chemiluminescence imaging, fluorescence resonance energy transfer (FRET) and UV-vis light absorbance. Spectroscopic detection methods include, but are not limited to, mass spectrometry, nuclear magnetic resonance (NMR) spectroscopy, Raman spectroscopy, and infrared spectroscopy. Electrostatic detection methods include, but are not limited to, gel-based techniques, such as, for example, gel electrophoresis (e.g., agarose gel or polyacrylamide gel electrophoresis). Gel electrophoresis methods can separate different nucleic acid molecules in a reaction mixture based on the molecular size of the nucleic acid molecules. The separation profile (e.g., sizes of various nucleic acids in a reaction mixture) can be used to identify nucleic acid molecules subject to gel electrophoresis according to their molecular sizes. Electrochemical detection methods include, but are not limited to, amperometry.

The following examples are offered for illustrative purposes only and are not intended to limit the scope of the present invention in any way. Indeed, various modifications of the invention in addition to those shown and described herein will become apparent to those skilled in the art from the foregoing description and fall within the scope of the appended claims.

### Example 1

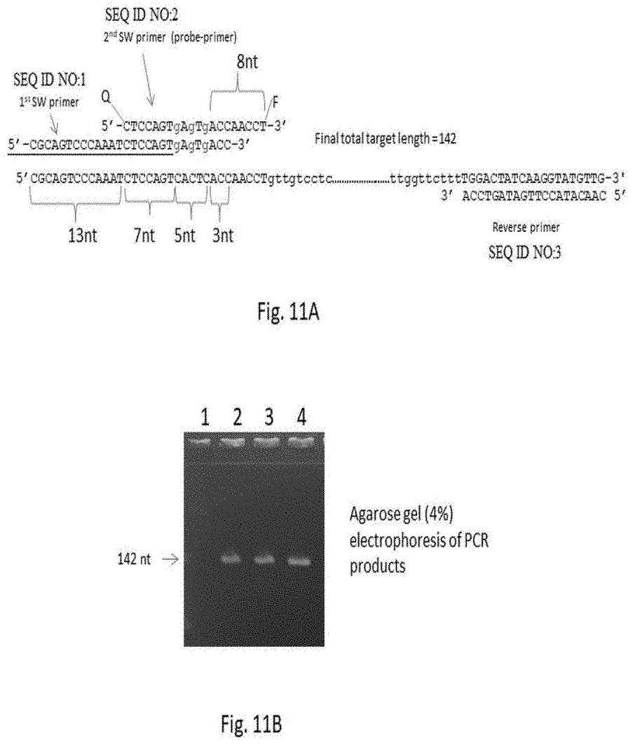

Single tube Nested PCR detection of Hepatitis B virus (HBV) DNA. This example describes experimental data for detecting the viral nucleic acids in samples with very low copy number of a pathogen. Saliva of individuals with hepatitis B are collected. Saliva of people with hepatitis B can contain the hepatitis B virus, but in very low concentrations compared with blood. In order to amplify a region of 155 nucleotides of HBV DNA, single-tube nested PCR reactions using primers 1st SW primer (SEQ ID NO: 1), 2nd SW primer (probe-primer, SEQ ID NO: 2) and reverse primer (SEQ ID NO: 3) are performed (as illustrated in FIG. 11A).

The 1st SW primer (SEQ ID NO: 1) comprises: (i) a 5′ anchor region which has a length of 13 nucleotides (nt), (ii) a 5′ recognition region has 7 nucleotides (nt), (iii) the first SW site comprises 5 nucleotides (nt), and (iv) a 3′ extension site has 3 nucleotides (nt). The 2nd SW primer (probe-primer, SEQ ID NO: 2) comprises: (i) a 5′ recognition region has 7 nucleotides (nt), (iii) the second SW site comprises 5 nucleotides (nt), and (iii) a 3′ extension site has 8 nucleotides (nt). The reverse primer is a standard oligonucleotide without modification.

Single tube Nested DNA amplification reactions are performed in home-made test tubes, each having a 50-ul reaction that includes: 20 mM Tris-HCl, pH 8.8, 2 mM MgSO4, 40 mM K2504, 0.1% Tween-20, 1 M Betaine, 200 nM dNTP, 0.06 U/μl DNA Pfu DNA polymerase, DNA template molecules, 0.3 μM reverse primer and 0.3 μM 2nd SW primer. Reactions (test-tubes) contain varying amounts of 1st SW primer and the results are shown in FIG. 11B. The 1st SW primer is added at a ratio to the 2nd SW primer: 0 (reaction-1), 0.5 (reaction-2), 1 (reaction-3) and 2 (reaction-4). The reactions are performed in a home-made convective thermal device (isothermal device), which has a temperature gradient from 55° C. to 95° C.

After reaction for 40 min, 10 μl of reaction product from each of the 4 test tubes is retrieved and subject to electrophoresis in a 4% agarose gel (as illustrated in FIG. 11B). Lane 1 shows the results obtained with a ratio (1st SW primer to 2nd SW primer) of 0, lane 2 shows the results obtained with a ratio of 0.5, lane 3 shows the results obtained with a ratio of 1, lane 4 shows the results obtained with a ratio of 2. No reaction product is detected in lane 1, which has no 1st SW primer. Lanes 2-4, all have single bands of the expected size of 142 nucleotides (nt). The results demonstrate that both 1st SW primer and 2 SW primer are needed for single tube nested PCR. Also, single-tube nested PCR of present disclosure can also detect low copy number of target nucleic acid molecules.

### Example 2

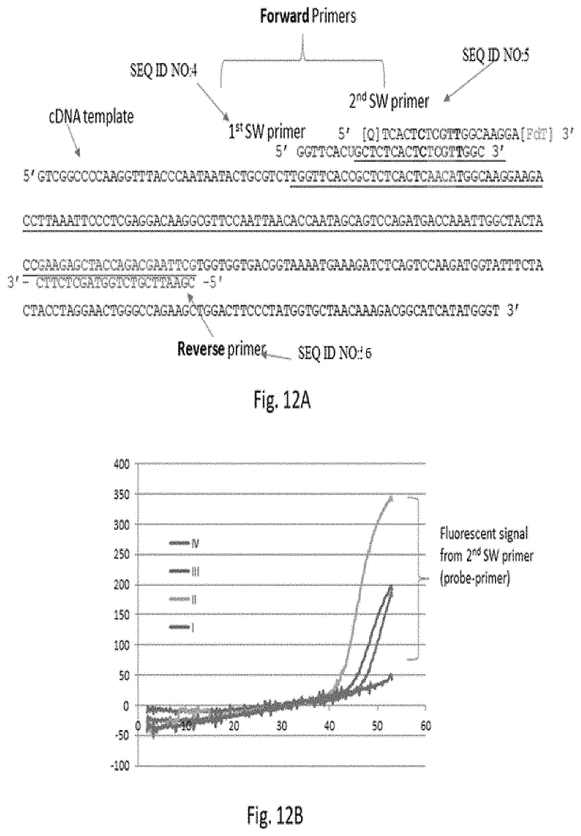

Single tube nested PCR detection of DNA derived from SARS-CoV-2 RNA. In order for detection of DNA derived from SARS-CoV-2 RNA, single-tube nested PCR reactions using primers 1st SW primer (SEQ ID NO: 4), 2nd SW primer (probe-primer, SEQ ID NO: 5) and reverse primer (SEQ ID NO: 6) are performed (as illustrated in FIG. 12A).

The 1st SW primer (SEQ ID NO: 4) comprises nucleobase U (uracil) as attenuating site so that DNA polymerase (Pfu polymerase) can stall at this site. The SW site which has length of 4 nucleotides. The 3′ end molecular moiety which is cleaved before extension by the 3′-5′ exonuclease activity of the DNA polymerase (Pfu type). The molecular moiety reduces nonspecific amplification. The 2nd SW probe primer (SEQ ID NO: 5) comprises: a 3′end labeled fluorescent dye called Fluorescein dT, which is cleaved before extension due to the 3′-5′ exonuclease activity of the DNA polymerase (Pfu type), a 5′ end labeled quencher, and the SW site which has length of 4 nucleotide. A reverse primer (SEQ ID NO: 6) is a standard oligonucleotide without modification.

Single tube Nested DNA amplification reactions are performed in 4 test tubes, each having a 50-ul reaction that includes: 20 mM Tris HCl, pH 8.8, 2 mM MgSO4, 40 mM 1(2504, 0.1% Tween 20, 1 M Betaine, 200 nM dNTP, 0.06 U/μl DNA Pfu type DNA polymerase, 1000 copies of DNA template molecules, 0.3 μM reverse primer (SEQ ID NO: 6) and 0.3 μM 2nd SW probe primer (SEQ ID NO: 5). 1st SW primer (SEQ ID NO: 4) is added to each of the 4 test tubes at the following concentration, respectively:

Test tube I: 0.0 μM

Test tube II: 0.2 μM

Test tube III: 0.4 μM

Test tube IV: 0.6 μM

The reactions are performed in a home-made convective thermal device (isothermal device), which has a temperature gradient from 55° C. to 95° C. Data is recorded in real-time by the instrument.

Results shows that in the absence of 1st SW primer (test tube I), 2nd SW probe primer was not able to amplify the target (as illustrated in FIG. 12B). Both the 1st SW primer and the 2nd SW probe primer are needed for single tube nested PCR. Fluorescent signals are generated from 2nd SW probe primer, depending on amplification from 1st SW primer. Weaker signals are produced in test tubes III and IV might be due to high concentration of 1st SW primer competing for the binding site with 2nd SW probe primer, resulting in lower amplification efficiency. The results demonstrate that method disclosed in the above embodiments can be optimized to effectively perform the single tube nested PCR. Based on the inflection points changes, this method can be used for quantitative detection pathogens.

### Example 3

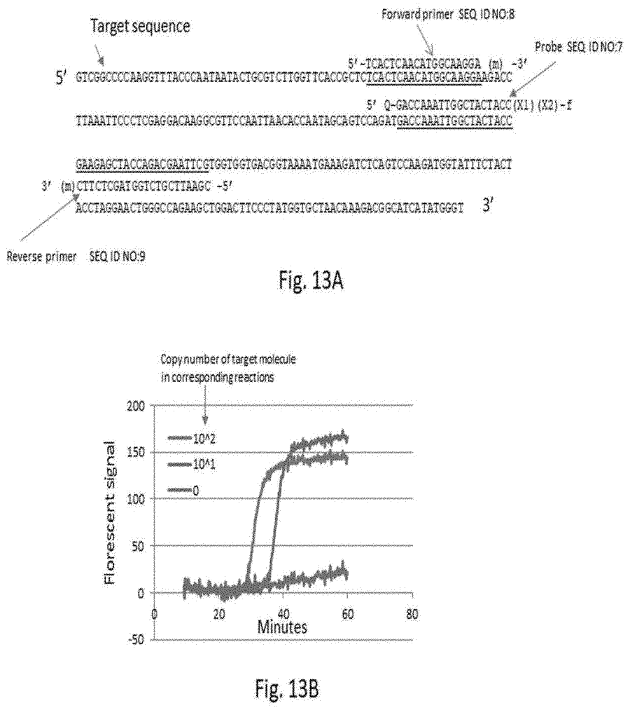

qPCR for the specific and/or quantitative detection of the SARS-CoV-2 gene. In order for detection of SARS-CoV-2 gene, quantitative PCR reactions using a probe (SEQ ID NO: 7), a forward primer (SEQ ID NO: 8) and a reverse primer (SEQ ID NO: 9) are performed. The sequence of the probe, forward primer and reverse primer as follows (as illustrated in FIG. 13A):

Probe (SEQ ID NO: 7):
5′-Q-GACCAAATTGGCTACTACCG(X1)(X2)-F-3′;
Forward primer (SEQ ID NO: 8):
5′-TCACTCAACATGGCAAGGA(m)-3′;
Reverse primer (SEQ ID NO: 9):
5′-CGAATTCGTCTGGTAGCTCTTC(m)-3′;

wherein, Q; quencher, F: fluorescent dye, X1 & X2: modified nucleotides or analogues as the attenuating site and m: 3′ non-complementary moiety.

Reaction setup: a 50 μL reaction containing the following: 20 mM Tris-HCl, pH 8.8, 0.1% Tween-20, 2 mM MgSO4, 30 mM 1(2504, 200 μM dNTP, 0.3 μM of each of the primers and the probe, 0.06 U/μl of Pfu-type DNA polymerase (with 3′-5′ exonuclease activity), target molecule at designated amounts. Reactions are performed in a home-made convective thermal device with top temperature set at 55° C., bottom temperature at 95° C.; fluorescent signals are read every 10 seconds.

Result shows the low copy numbers of DNA derived from a SARS-CoV-2 RNA can be detected in about 30 min (as illustrated in FIG. 13B).

1. A method comprising:
a. assembling a reaction mixture comprising:
I. a target molecule comprising a nucleic acid sequence of interest;
II. a primer set comprises a pair of amplification primers;
III. a probe comprising: (i) an attenuating site, (ii) a first label in a non 3′ site, and (iii) a second label at the 3′ end;
IV. a polymerase with a 3′-5′ exonuclease activity;
b. conducting an amplification reaction of the target molecule comprising the nucleic acid sequence of interest using the reaction mixture;
wherein the probe and the polymerase with 3′-5′ exonuclease activity is configured to enable the specific and/or quantitative detection of the nucleic acid sequence of interest.

2. The method of claim 1, wherein the second label at the 3′ end of the probe is cleavable effectively using the 3′-5′ exonuclease activity of the polymerase.

3. The method of claim 1, wherein the first label and the second label comprise a fluorescent dye-quencher pair or similar thereof.

4. The method of claim 1, wherein the primer set comprises a first primer and a second primer, wherein the first primer and the second primer are complementary to the target molecule comprising the nucleic acid sequence of interest.

5. The method of claim 1, wherein the attenuating site is located between the center of the probe and the second label and comprises at least 1 to 10 units, comprising a natural nucleotide, a non-natural nucleotide, an abasic site, a spacer, a fluorescent label-modified nucleotide, an atypical nucleotide comprised of deoxyuridine, a chemically synthesized nucleotide or combination thereof.

6. The method of claim 1, wherein the nucleic acid sequence of interest comprises a SARS-CoV-2 sequence.

7. (canceled)

8. The method of claim 1, wherein the specific and/or quantitative detection of the nucleic acid sequence of interest comprises:
i. annealing an amplification primer to a strand of the target molecule comprising the nucleic acid sequence of interest;
ii. amplifying the two strands of the target molecule between the first and second amplification primer sites in the presence of the polymerase;
iii. hybridizing the probe to a strand of the target molecule to form a probe:target duplex;
iv. detecting florescence emission after cleavage of a label off the probe using the 3′-5′ exonuclease activity of the polymerase.

9. The method of claim 1, wherein a 3′ end of a primer of the primer set comprises a molecular moiety, wherein the molecular moiety is non-complementary to a target nucleic acid sequence of interest.

10. The method of claim 9, wherein the molecular moiety is configured to be cleaved by the 3′-5′ exonuclease activity of the DNA polymerase prior to extension of a primer using the polymerase.

11-14. (canceled)

15. A method comprising:
(a) assembling a reaction mixture comprising:
I. a target molecule comprising a nucleic acid sequence of interest;
II. a set of oligonucleotides comprising:
1) a 1^{st} SW (selective wobble) primer comprising a 1^{st} SW site;
2) a 2^{nd} SW primer comprising a 2^{nd} SW site;
3) at least a third primer;
4) a probe comprising: (i) an attenuating site, (ii) a first label in a non 3′ site and (iii) a second label at the 3′ end;
III. a polymerase with 3′-5′ exonuclease activity;
(b) conducting an amplification reaction of the target molecule comprising the nucleic acid sequence of interest using the reaction mixture;
(c) detecting or amplifying the target molecule comprising the nucleic acid sequence of interest or variants thereof present in the target molecule;
wherein the SW sites are configured to enable non-disrupted nested amplification and quantification of the target molecule comprising the nucleic acid sequence of interest.

16-21. (canceled)

22. The method of claim 15, wherein amplification of the target molecule comprising the nucleic acid sequence of interest comprises a SW method comprising:
i. extending the 1^{st} SW primer using the polymerase to generate a mutated strand of the target molecule;
ii. generating a mutated complementary strand from the mutated strand using the third primer;
iii. amplifying the mutated complementary strand and the mutated strand using the 2^{nd} SW primer and the third primer, wherein the 2^{nd} SW primer is configured to anneal to the mutated complementary strand.

23. The method of claim 15, wherein the second label at the 3′ end label of the probe is cleavable effectively using the 3′-5′ exonuclease activity of the polymerase.

24. The method of claim 15, wherein the first label and the second label of the probe comprises a fluorescent dye-quencher pair or similar thereof.

25-45. (canceled)

46. A kit comprising:
a) a primer set comprises a pair of amplification primers;
b) a probe comprising: (i) an attenuating site, (ii) a first label in a non 3′ site and (iii) a second label at the 3′ end;
c) a polymerase with a 3′-5′ exonuclease activity;
wherein the kit is configured to detect a target molecule comprising a nucleic acid sequence of interest or variants thereof.

47. The kit of claim 46, wherein the second label at the 3′ end label of the probe is cleavable effectively using the 3′-5′ exonuclease activity of the polymerase.

48. The kit of claim 46, wherein the first label and the second label comprise a fluorescent dye-quencher pair or similar thereof.

49. The kit of claim 46, wherein the attenuating site is located between the center and the second label and comprises at least 1 to 10 units, selected from the group of a natural nucleotide, a non-natural nucleotide, an abasic site, a spacer, a fluorescent label-modified nucleotide, an atypical nucleotide comprised of deoxyuridine, a chemically synthesized nucleotide or combination thereof.

50. The kit of claim 46, wherein a 3′ end of the primer set comprises a molecular moiety, wherein the molecular moiety is non-complementary to a target molecule sequence of interest.

51-58. (canceled)


SEQUENCE LISTING (11 sequences)

SEQ ID NO: 1 - DNA, 28 bp, Hepatitis B virus
cgcagtcccaaatctccagtgagtgacc

SEQ ID NO: 2 - DNA, 20 bp, Hepatitis B virus
ctccagtgagtgaccaacct

SEQ ID NO: 3 - DNA, 20 bp, Hepatitis B virus
caacataccttgatagtcca

SEQ ID NO: 4 - DNA, 25 bp, SARS-CoV-2
ggttcacgctctcactctcgttggc

SEQ ID NO: 5 - DNA, 19 bp, SARS-COV-2
tcactctcgttggcaagga

SEQ ID NO: 6 - DNA, 22 bp, SARS-COV-2
cgaattcgtctggtagctcttc

SEQ ID NO: 7 - DNA, 18 bp, SARS-COV-2
accaaattggctactacc

SEQ ID NO: 8 - DNA, 20 bp, SARS-COV-2
tcactcaacatggcaaggam

SEQ ID NO: 9 - DNA, 23 bp, SARS-COV-2
gcttaagcagaccatcgagaagm

SEQ ID NO: 10 - DNA, 155 bp, Hepatitis B virus
cgcagtcccaaatctccagtcactcaccaacctgttgtcctccaatttgtcctggttatcgctggatgtgtctgcggcgttttatcatcttcctctgcatcctgctgctatgcctcatcttcttgttggttcttctggactatcaaggtatgttg

SEQ ID NO: 11 - DNA, 280 bp, SARS-COV-2
gtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggt